(19) United States
(12) Patent Application Publication
(10) Pub. No.: US 2025/0325537 A1
(43) Pub. Date: Oct. 23, 2025

(54) IONOPHORIC COPPER-CHELATORS IN COMBINATION WITH MAPK INHIBITORS FOR USE IN TREATMENT OF CANCER

(71) Applicant: UNIVERSITÄT ZÜRICH, Zürich (CH)

(72) Inventors: Ossia-Margarita EICHHOFF, Zürich (CH); Mitch LEVESQUE, Zürich (CH); Reinhard DUMMER, Zürich (CH); Annalisa SALTARI, Zürich (CH); Andreas DZUNG, Zürich (CH); Luzia SEILER, Zürich (CH); Corinne Isabelle STOFFEL, Zürich (CH)

(73) Assignee: UNIVERSITÄT ZÜRICH, Zürich (CH)

(21) Appl. No.: 18/873,352

(22) PCT Filed: Jun. 12, 2023
(86) PCT No.: PCT/EP2023/065676

§ 371 Date: Dec. 10, 2024

(30) Foreign Application Priority Data
Jun. 10, 2022 (EP) 22178503.3

(51) Int. Cl. A61K 31/4745; A61K 31/166; A61K 31/404; A61K 31/4184; A61K 31/437; A61K 31/4523; A61K 31/506; A61K 31/519; A61K 31/5377; A61P 35/00

(52) U.S. Cl. CPC A61K 31/4745; A61K 31/166; A61K 31/404; A61K 31/4184; A61K 31/437; A61K 31/4523; A61K 31/506; A61K 31/519; A61K 31/5377; A61P 35/00

(57) ABSTRACT

The present invention relates to the use of neocuproine, elesclomol, disulfiram and/or dithiocarbamate for use in treatment of cancer, optionally in combination with MAPK-pathway inhibitors in cancers carrying mutations that are recognized as MAPK-pathway activating mutations.

This application claims the right of priority of European Patent Application EP22178503.3 filed 10 Jun. 2022, which is incorporated by reference herein.

### FIELD OF THE INVENTION

The present invention relates to the use of neocuproine, elesclomol, disulfiram and/or dithiocarbamate for use in treatment of cancer, optionally in combination with one or more MAPK-pathway inhibitors in cancers carrying mutations that are recognized as MAPK-pathway activating mutations.

### BACKGROUND OF THE INVENTION

### Oncogenic Mutations in Solid Tumours and MAPK Inhibitors

Despite numerous powerful and successful studies on cancer, the rapid increase in cancer prevalence and cancer mortality rates is one of the biggest problems worldwide. The causes for these phenomena include environmental pollution, population growth and aging. In both genders the most lethal cancer types among solid tumors are lung cancer (with 14% mortality rate of the total cases) being the most prominent reason for death, followed by female breast cancer (13.6%), colon cancer (9%), liver cancer (8.2%) and prostate cancer (7.7%) (www.gco.iarc.fr). In most cancer patients, chemotherapy or radiotherapy are the primary approaches, together with excision of solid and local tumors. However, in the last two decades, cancer research has led to the development of small molecule therapies targeting activated kinases and -pathways. This is due to massive sequencing of cancer tissues revealing druggable common oncogenic driver mutations. Mutations in RAS oncogene (HRAS, NRAS, and KRAS) are among the most common ones. RAS is a small GTPase which is a regulator for important cellular functions such as proliferation, cell migration, programmed cell death (apoptosis) and survival. These cellular events need to be controlled tightly, and dysregulation caused by activating mutations can hyper-activate downstream signaling cascades and lead to malignant transformation, tumor growth and metastasis. For example, activating mutation in RAS, mainly involving amino acids G12, G13 or Q61, which lead to an over-activated RAS kinase, will hyper-activate the downstream signaling cascade called MAPK (mitogen-activated protein kinase) pathway, which normally controls expression of genes involved in proliferation, migration, invasion and cell fate determination. In contrast to other oncogenic activating mutations, there is no kinase inhibitor that directly targets RAS kinase. Therefore, downstream kinase inhibitors like panRAF (e.g. Belvarafenib, Naporafenib) or MEK inhibitors (e.g. Binimetinib, Trametinib etc) are used in such cases. Furthermore, mutations in the BRAF kinase or receptor-tyrosine kinases like cKit (v-kit proto-oncogen) or EGFR (epidermal-growth factor receptor) also lead to activated MAPK signaling. Here, direct kinase inhibitors are available (e.g. Dabrafenib, Encorafenib, Imatinib, Sunitinib). Despite extensive clinical research for the use of kinase inhibitors for solid tumors, there is still a lack of outstanding clinical successes, especially for MAPK-pathway inhibitor (MEKi, BRAFi, panRAFi) or tyrosine kinase inhibitor monotherapy, and relapse is mostly observed. Hence, a high medical need exists for novel combinatorial treatments that can increase the efficiency of kinase inhibitors.

### The Role of Copper and Copper-Chelators in Cancer and in the Formation of Reactive Oxygen Species

Copper (Cu) is an essential trace element that is indispensable for life. This metal serves as catalytic and structural cofactor for enzymes involved in many physiological processes such as energy generation, iron acquisition, oxygen transport, cellular metabolism, signal transduction, and blood clotting. The homeostatic balance of bioavailable copper in the human body has shown to effect tumor growth and copper levels are elevated in cancer patients. Brady et al. showed that copper is a coactivator of the MAPK pathway by interacting with MEK1/2, enabling the phosphorylation MAPK down-stream target Erk. Moreover, the role of Ctrl, the transporter responsible for copper ion-influx into the cells was studied by interrupting its expression, which lead to decreased oncogenic BRAF signaling, thus highlighting the role of copper in MAPK-driven melanoma cells. Copper chelators used in the treatment of Wilsons's disease were suggested to be beneficial for cancer patients (Trientin). It was also shown that copper availability affects the growth of pancreatic tumors, and that intracellular copper uptake regulates a cancer cell metabolic phenotype shown in the presence of the copper chelator tetrathiomolybdate.

On the other hand, and in contrast to Cu chelators, the chemical class of Cu ionophores chelate metal ions in the extracellular space and transport them through biological membranes thus increasing the intracellular copper levels. In the case of Cu binding for example the reduction of bound Cu(II) to Cu(I) and the release of Cu ions from the ionophore will lead to the production of reactive-oxygen species (ROS), toxic to cancer cells. Copper ionophores are Cu dependent and in combination with Cu chelators (e.g. Trientin or tetrathiomolybdate) lose or decrease their toxic activity on cancer cells. Some copper chelators have been already studied extensively also for their clinical relevance. The most well-known are: Disulfiram and dithiocarbamates; clioquinol and hydroxyquinolines; elesclomol, and neocuproine.

### Neocuproine

Neocuproine, also known as 2,9-dimethyl-1,10-phenanthroline, is a member of the family of phenantrolines, and a specific Cu(I) chelator. Neocuproine is known as a copper chelating agent which upon binding of Cu(II) rapidly reduced to Neocuproine-Cu(I) complexes. Although these complexes are described as stable, it was observed that Neocuproine-Cu(I), in orchestra with the antioxidant-defense molecule Glutathione, induces DNA scission through oxidative mechanisms.

When neocuproine is given in solution together with e.g. CuSO₄, Cu(II)-neocuproine complexes are formed and it was noticed that there is synergistic cytotoxic effects when CuSO₄ is added together with neocuproine on L1210 mouse lymphocytic leukemia cells. Neocuproine complexed with Cu(I) did show tumor promoting effects in a B16 mouse melanoma model, and also resulted in enhanced tumor pigmentation. On the other hand, when neocuproine was added into the water of adult zebrafish, it was shown to decrease pigmentation and to induce melanocytes death. Byrnes et al. showed that neocuproine inhibited the growth of Ehrlich ascites tumor cells and noticed the synergistic effect in inhibition of cellular growth when neocuproine was given together with CuCl₂. The cytotoxic and DNA damaging cellular effects is thought to be induced by hydroxyl radicals (oxidative stress) which are formed during the reduction of Copper to Cu(I) when bound to neocuproine while internalized into the cells. This effect was also demonstrated in Escherichia coli where a synergistic effect of neocuproine with hydrogen peroxide resulted in increased number of DNA strand breaks and oxidative stress. Moreover, Cu(I) was suggested to be important for proteasome function, and it was shown that the presence of Neocuproine impairs that function, so it can be said that Neocuproine induces proteasome inhibition, which might be another mechanism of action, independent of its ROS inducing function, and which could inhibit cancer cells viability.

### Elesclomol

Elesclomol is a bis(thiohydrazide) amide, and similar to neocuproine, a compound that binds copper. Elesclomol was identified through an original cell-based multidrug resistance modulators screen where it was synthesised from a given parental compound library (Chen, S., et al., Bioorg Med Chem Lett, 2013. 23(18): p. 5070-6). It was further developed by Synta Pharmaceuticals and previously tested in several clinical trials in a combination treatment with paclitaxel for anticancer activity of solid tumours including metastatic melanoma (Berkenblit, A., et al., Clin Cancer Res, 2007. 13(2 Pt 1): p. 584-90; O'Day, S., et al., J Clin Oncol, 2009. 27(32): p. 5452-8, O'Day, S. J., et al., J Clin Oncol, 2013. 31(9): p. 1211-8.). Mechanistically, elesclomol binds Cu(II) in the extracellular environment and forms a membrane permeable complex, which upon entering the mitochondria releases copper after it is reduced to Cu(I). Cu(I) released in the mitochondria can react with molecular oxygen to generate ROS, which can cause unmitigated oxidative stress and apoptotic death of cancer cells.

### Disulfiram (DSF) and Dithiocarbamate (DDC)

DSF is an orally administered drug for the treatment of alcoholism. The drug inhibits the enzyme aldehyde dehydrogenase, which is an enzyme important for the alcohol metabolism in the liver. The combination of DSF and alcohol therefore leads to alcohol intolerance.

Another chemical function of DSF is the conversion to DDC in the stomach, which creates copper-DDC complexes. These complexes are known to increase the intracellular Cu levels and therefore DDC is classified as an ionophoric copper-chelator. Also, its intracellular action is abolished when given in combination with non-permeable Cu-chelators (e.g. Trientine). There are several clinical studies ongoing that investigate DSF for the treatment of cancer, but none of them is suggesting the combination of disulfiram in combination with binimetinib for the treatment of metastatic melanoma (Babak, M. V. et al., Biomedicines, 2021. 9(8)).

### Reactive Oxygen Species in the Treatment of Cancer

ROS (reactive oxygen species) is a collective term used to describe chemical species that are produced as byproducts of normal oxygen metabolism and include superoxide anion (O2-), hydrogen peroxide (H₂O₂), and the hydroxyl radical. Upregulation of ROS is often associated with chemotherapy or kinase inhibitor treatment of tumor cells. For example, it was demonstrated that oncogenic BRAF mutations, resulting in hyper-activation of MAPK signaling, maintain a glycolytic phenotype in melanoma, thus delivering anti-oxidant defense mechanism via the pentose phosphate pathway to insure survival of cancer cells and linking glycolysis with intracellular ROS levels. In contrast to the Warburg hypothesis, according to which tumors produce large portion of energy (ATP) through glycolysis, studies have shown that melanomas, when treated with MAPK pathways inhibitors, where glycolysis is decreased, have elevated level of oxygen consumption and therefore high oxidative phosphorylation rates (OXPHOS), resulting in elevated beta-oxidation and altered generation of superoxide anions. Moreover, metastasizing cells have intrinsic high ROS levels due to low glycolysis turnover and these cells are highly depending on NADPH-detoxifying enzymes. Any misbalance of NADPH recycling enzymes or other interventions, which result in elevated ROS levels, significantly decrease metastasis. In colorectal cancer resistance to chemotherapies leading to a transient diapause-like dedifferentiated cell state, which is maintained by upregulation of autophagy and decreased transcription but display high intracellular ROS levels. Non-small cell lung cancer (NSCLC) tumors implicate heterogeneity within cancer cell populations as a response to drug treatments. Drug-tolerant persister cells survive treatment with inhibitor, but are slow cycling. These upregulate insulin growth factor (IGF) signaling and alter chromatin state by histone demethylase activity. As a result, intracellular ROS levels are upregulated. In breast cancer, resistance to the tyrosine kinase inhibitor lapatinib is achieved through metabolic adaptation favoring mitochondrial energy metabolism through increased glutamine metabolism, resulting in ROS production. Moreover, targeting enhanced oxidative phosphorylation (OXPHOS), where elevated ROS is seen as a by-product, inhibits bone metastasis in triple-negative breast cancer (TNBC) cells.

In non-cancer cells, ROS are produced at low concentration and therefore effectively neutralized by the potent antioxidant system of the cells. Therefore, targeting ROS in aggressive cancer cells with already elevated ROS levels could be a promising treatment option). In summary, the combination of MAPK inhibitors, which have been shown to target tumor growth and ionophoric copper chelators, which induce ROS effectively in cancer cells, have a beneficial effect on tumor cell growth, metastasis and apoptosis.

### DESCRIPTION OF THE INVENTION

### Summary of the Invention

In one aspect, the invention relates to the use of neocuproine in treatment of cancer characterized by cells having an oncogenic mutation associated with significantly increased occurrence of malignant tumours, selected from the group consisting of: an NRAS mutation, a KRAS mutation, an HRAS mutation, a BRAF mutation, a cKIT mutation, an NF1 loss of function mutation.

In another aspect, the invention relates to a combination medicament comprising neocuproine and a MAPK-pathway inhibitor. This combination medicament may be used in treatment of cancer.

A further aspect of the invention relates to a combination medicament comprising neocuproine and a tyrosine kinase inhibitor. This combination medicament may be used in treatment of cancer.

In yet another aspect, the invention relates to the use of a compound selected from the group comprising neocuproine, elesclomol, disulfiram, and dithiocarbamate for use in treatment of cancer.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. cancer) refers in one embodiment to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

In the context of the present specification, the term resistant to treatment shall mean that the cells that are thus characterized do not respond to the indicated treatment.

In the context of the present specification, the term refractory to treatment shall mean that the cells that are thus characterized do not respond as well to the indicated treatment as to achieve the desired therapeutic effect expected for fully responsive cells.

In the context of the present specification, the term gene refers to the DNA sequence encoding a particular protein. When the term mutation refers to a protein name, it is understood that the respective DNA sequence harbours the mutation which leads to a change in the amino acid sequence of the protein.

In the context of the present specification, the term mutation refers to a non-silent change in the nucleic acid sequence of a gene. In certain embodiments, this change is a substitution of one or more base pairs.

In the context of the present specification, the term constitutively activating refers to an enzyme which is active even without activation of the upstream signalling cascade.

In the context of the present specification, the term loss of function refers to a protein which is not able to fulfil its native function to a similar extend as the wildtype gene.

The abbreviations of genes are listed below with their identifiers:
KRAS: NCBI Gene: 3845, Ensembl: ENSG00000133703
HRAS: NCBI Gene: 3265, Ensembl: ENSG00000174775
NRAS: NCBI Gene: 4893, Ensembl: ENSG00000213281
BRAF: NCBI Gene: 673, Ensembl: ENSG00000157764
cKIT: NCBI Gene: 3815, Ensembl: ENSG00000157404
NF1: NCBI Gene 4763, Ensembl ENSG00000196712

### Intra-Tumor Heterogeneity and Treatment

In recent years, targeting the MAPK-pathway has shown dramatic clinical effects. In melanoma for example, combined MAPK-pathway inhibitor therapy (BRAF inhibition+MEK inhibition), led to improvements in 5-year overall survival in patients with BRAF mutated tumours. Due to the lack of durably efficient targeted therapies against mutated RAS kinase proteins, immunotherapies are the recommended first-line treatment for this patient cohort. MEK inhibitor monotherapy in NRAS mutated melanoma can prolong progression-free survival and was suggested as a treatment option after the failure of immunotherapy. Unfortunately, targeting MAPK-signaling in NRAS-mutated melanoma is only beneficial to a small subset of patients (response rate for Binimetinib is 15-20%). Moreover, combined MAPK-pathway inhibition frequently results in resistance formation. One major obstacle in the treatment of solid tumors in general is the intra-tumor heterogeneity, which is derived from transcriptional cell plasticity (also called by the term epithelial-mesenchymal transition).

Based on the above-mentioned state of the art, the objective of the present invention is to provide means and methods for the therapy of cancers with activation of MAPK signalling. This objective is attained by the claims of the present specification.

### DETAILED DESCRIPTION OF THE INVENTION

The present invention focuses on the treatment of tumor cell heterogeneity by combining inhibitors of the MAPK signaling pathway (MAPK-pathway) or tyrosine kinase inhibitors and small molecules which are able to induce ROS, known to be toxic for resistant cancer cells especially. The inventors show that the combination of a MAPK inhibitor or tyrosine kinase inhibitors and a ROS inducer lead to greater tumor growth inhibition and also can prolong the survival in vivo when compared to MAPK inhibition or tyrosine kinase inhibition alone.

A first aspect of the invention relates to neocuproine for use in treatment of cancer.

An alternative of the first aspect of the invention relates to elesclomol for use in treatment of cancer.

An alternative of the first aspect of the invention relates to disulfiram for use in treatment of cancer.

An alternative of the first aspect of the invention relates to dithiocarbamate for use in treatment of cancer.

In certain embodiments, the cancer is characterized by cells having a constitutively activating NRAS mutation. In certain embodiments, the NRAS mutation is selected from the group comprising NRAS Q61K, NRAS Q61L, NRAS Q61R, NRAS Q61H, or NRAS G12A.

In certain embodiments, the NRAS mutation is selected from the group of an NRAS Q61 mutation, an NRAS G12 mutation and an NRAS G13 mutation. In certain embodiments, the NRAS mutation is selected from the group of NRAS Q61R, NRAS Q61K, NRAS Q61L, and NRAS Q61H, NRAS G12D, NRAS G12S, NRAS G12D, NRAS G12C, NRAS G12V, NRAS G12A, NRAS G13D, NRAS G13R, NRAS G13V, NRAS G13C. In certain embodiments, the NRAS mutation is selected from the group of NRAS Q61K, NRAS Q61L, NRAS Q61R, NRAS Q61H, and NRAS G12A.

In certain embodiments, the cancer is characterized by cells having a constitutively activating KRAS mutation.

In certain embodiments, the KRAS mutation is selected from the group of a KRAS Q61 mutation, a KRAS G12 mutation, and a KRAS G13 mutation. In certain embodiments, the KRAS mutation is selected from the group of KRAS Q61R, KRAS Q61K, KRAS Q61 L, KRAS Q61H, KRAS G13D, KRAS G13R, KRAS G13V, KRAS G13C, KRAS G12D, KRAS G12S, KRAS G12D, KRAS G12C, KRAS G12V, and KRAS G12A.

In certain embodiments, the cancer is characterized by cells having a constitutively activating HRAS mutation.

In certain embodiments, the HRAS mutation is selected from the group of an HRAS Q61 mutation, an HRAS G12 mutation, and an HRAS G13 mutation. In certain embodiments, the HRAS mutation is selected from the group of HRAS Q61R, HRAS Q61K, HRAS Q61 L, HRAS Q61H, HRAS G12D, HRAS G12S, HRAS G12D, HRAS G12C, HRAS G12V, HRAS G12A, HRAS G13D, HRAS G13R, HRAS G13V, and HRAS G13C.

In certain embodiments, the cancer is characterized by cells having a constitutively activating BRAF mutation. In certain embodiments, the BRAF mutation is selected from the group comprising BRAF V600E, BRAF V600K, BRAF V600D or BRAF V600R.

In certain embodiments, the cancer is characterized by cells having a constitutively activating cKIT mutation. In certain embodiments, the cKIT mutation is selected from the group comprising cKIT K642E, cKIT L576P, cKIT V559A and cKIT W557R.

In certain embodiments, the cancer is characterized by cells characterized by a mutation in two genes of the group comprising NRAS, BRAF, and cKIT. In certain embodiments, the cancer is characterized by cells characterized by a mutation in NRAS, and BRAF. In certain embodiments, the cancer is characterized by cells characterized by a mutation in NRAS, and cKIT. In certain embodiments, the cancer is characterized by cells characterized by a mutation in BRAF and cKIT.

In certain embodiments, the said cancer is characterized by cells characterized by a mutation in all three genes of the group comprising NRAS, BRAF, and cKIT.

In certain embodiments, the cancer is characterized by cells that are resistant or refractory to treatment with a MEK inhibitor. In certain embodiments, the MEK inhibitor is selected from the group consisting of trametinib, binimetinib, cobimetinib and selumetninib.

In certain embodiments, the cancer is characterized by cells that are resistant to treatment with a BRAF inhibitor. In certain embodiments, the BRAF inhibitor is selected from the group consisting of vemurafenib, dabrafenib and encorafenib.

In certain embodiments, the cancer is characterized by cells that are resistant to treatment with a cKIT inhibitor. In certain embodiments, the cKIT inhibitor is selected from the group comprising imatinib, sunitinib, desatinimb or nilotinib.

In certain embodiments, the cancer is characterized by cells
a. having a BRAF mutation, and
b. are resistant to treatment with a BRAF inhibitor.

In certain embodiments, the cancer is characterized by cells
a. having an NRAS mutation, and
b. are resistant to treatment with a MEK inhibitor.

In certain embodiments, the cancer is characterized by cells
a. having a cKIT mutation, and
b. are resistant to treatment with a cKIT inhibitor.

A second aspect of the invention relates to a combination medicament (and to methods of manufacture of such combination medicament) comprising or essentially consisting of
a. neocuproine; and
b. a compound selected from the group comprising vemurafenib, encorafenib, dabrafenib, trametinib, binimetinib, cobimetinib, mirdametinib, selumetinib, imatinib, sunitinib, desatinib and nilotinib;
   for use in treatment of cancer.

This aspect also encompasses the use of neocuproine in treatment of cancer as characterized in detail herein, in co-administration to a patient treated concomitantly with
a BRAF inhibitor, particularly a BRAF inhibitor selected from the group consisting of vemurafenib, dabrafenib and encorafenib;
a MEK inhibitor, particularly a MEK inhibitor selected from the group of trametinib, binimetinib, cobimetinib and selumetninib,
a cKIT inhibitor, particularly a cKIT inhibitor selected from the group comprising imatinib, sunitinib, desatinimb or nilotinib.

Likewise, this aspect encompasses the use of
a BRAF inhibitor, particularly a BRAF inhibitor selected from the group consisting of vemurafenib, dabrafenib and encorafenib;
a MEK inhibitor, particularly a MEK inhibitor selected from the group of trametinib, binimetinib, cobimetinib and selumetninib,
a cKIT inhibitor, particularly a cKIT inhibitor selected from the group comprising imatinib, sunitinib, desatinimb or nilotinib.
   in treatment of cancer as characterized in detail herein, in co-administration to a patient treated concomitantly with neocuproine.

An alternative of the second aspect of the invention relates to a combination medicament (and to methods of manufacture of such combination medicament) comprising or essentially consisting of
a. elesclomol; and
b. a compound selected from the group comprising vemurafenib, encorafenib, dabrafenib, trametinib, binimetinib, cobimetinib, mirdametinib, selumetinib, imatinib, sunitinib, desatinib and nilotinib;
   for use in treatment of cancer.

This aspect also encompasses the use of elesclomol in treatment of cancer as characterized in detail herein, in co-administration to a patient treated concomitantly with
a BRAF inhibitor, particularly a BRAF inhibitor selected from the group consisting of vemurafenib, dabrafenib and encorafenib;
a MEK inhibitor, particularly a MEK inhibitor selected from the group of trametinib, binimetinib, cobimetinib and selumetninib,
a cKIT inhibitor, particularly a cKIT inhibitor selected from the group comprising imatinib, sunitinib, desatinimb or nilotinib.

Likewise, this aspect encompasses the use of
a BRAF inhibitor, particularly a BRAF inhibitor selected from the group consisting of vemurafenib, dabrafenib and encorafenib;
a MEK inhibitor, particularly a MEK inhibitor selected from the group of trametinib, binimetinib, cobimetinib and selumetninib,
a cKIT inhibitor, particularly a cKIT inhibitor selected from the group comprising imatinib, sunitinib, desatinimb or nilotinib.
   in treatment of cancer as characterized in detail herein, in co-administration to a patient treated concomitantly with elesclomol.

An alternative of the second aspect of the invention relates to a combination medicament (and to methods of manufacture of such combination medicament) comprising or essentially consisting of
a. disulfiram; and
b. a compound selected from the group comprising vemurafenib, encorafenib, dabrafenib, trametinib, binimetinib, cobimetinib, mirdametinib, selumetinib, imatinib, sunitinib, desatinib and nilotinib;
   for use in treatment of cancer.

This aspect also encompasses the use of disulfiram in treatment of cancer as characterized in detail herein, in co-administration to a patient treated concomitantly with
a BRAF inhibitor, particularly a BRAF inhibitor selected from the group consisting of vemurafenib, dabrafenib and encorafenib;
a MEK inhibitor, particularly a MEK inhibitor selected from the group of trametinib, binimetinib, cobimetinib and selumetninib,
a cKIT inhibitor, particularly a cKIT inhibitor selected from the group comprising imatinib, sunitinib, desatinimb or nilotinib.

Likewise, this aspect encompasses the use of
a BRAF inhibitor, particularly a BRAF inhibitor selected from the group consisting of vemurafenib, dabrafenib and encorafenib;
a MEK inhibitor, particularly a MEK inhibitor selected from the group of trametinib, binimetinib, cobimetinib and selumetninib,
a cKIT inhibitor, particularly a cKIT inhibitor selected from the group comprising imatinib, sunitinib, desatinimb or nilotinib.
   in treatment of cancer as characterized in detail herein, in co-administration to a patient treated concomitantly with disulfiram.

Yet another alternative of the second aspect of the invention relates to a combination medicament (and to methods of manufacture of such combination medicament) comprising or essentially consisting of
a. dithiocarbamate; and
b. a compound selected from the group comprising vemurafenib, encorafenib, dabrafenib, trametinib, binimetinib, cobimetinib, mirdametinib, selumetinib, imatinib, sunitinib, desatinib and nilotinib;
   for use in treatment of cancer.

This aspect also encompasses the use of dithiocarbamate in treatment of cancer as characterized in detail herein, in co-administration to a patient treated concomitantly with
a BRAF inhibitor, particularly a BRAF inhibitor selected from the group consisting of vemurafenib, dabrafenib and encorafenib;
a MEK inhibitor, particularly a MEK inhibitor selected from the group of trametinib, binimetinib, cobimetinib and selumetninib,
a cKIT inhibitor, particularly a cKIT inhibitor selected from the group comprising imatinib, sunitinib, desatinimb or nilotinib.

Likewise, this aspect encompasses the use of
a BRAF inhibitor, particularly a BRAF inhibitor selected from the group consisting of vemurafenib, dabrafenib and encorafenib;
a MEK inhibitor, particularly a MEK inhibitor selected from the group of trametinib, binimetinib, cobimetinib and selumetninib,
a cKIT inhibitor, particularly a cKIT inhibitor selected from the group comprising imatinib, sunitinib, desatinimb or nilotinib.
   in treatment of cancer as characterized in detail herein, in co-administration to a patient treated concomitantly with dithiocarbamate.

An alternative of the second aspect of the invention relates to a combination medicament (and to methods of manufacture of such combination medicament) comprising or essentially consisting of
a. neocuproine; and
b. a second compound, wherein the second compound is a MAPK-pathway inhibitor;
   for use in treatment of cancer.

An alternative of the second aspect of the invention relates to a combination medicament (and to methods of manufacture of such combination medicament) comprising or essentially consisting of
a. elesclomol; and
b. a second compound, wherein the second compound is a MAPK-pathway inhibitor;
   for use in treatment of cancer.

An alternative of the second aspect of the invention relates to a combination medicament (and to methods of manufacture of such combination medicament) comprising or essentially consisting of
a. disulfiram; and
b. a second compound, wherein the second compound is a MAPK-pathway inhibitor;
   for use in treatment of cancer.

An alternative of the second aspect of the invention relates to a combination medicament (and to methods of manufacture of such combination medicament) comprising or essentially consisting of
a. dithiocarbamate; and
b. a second compound, wherein the second compound is a MAPK-pathway inhibitor;
   for use in treatment of cancer.

An alternative of the second aspect of the invention relates to a combination medicament (and to methods of manufacture of such combination medicament) comprising or essentially consisting of
a. neocuproine; and
b. a second compound, wherein the second compound is a tyrosine kinase inhibitor; for use in treatment of cancer.

An alternative of the second aspect of the invention relates to a combination medicament (and to methods of manufacture of such combination medicament) comprising or essentially consisting of
a. elesclomol; and
b. a second compound, wherein the second compound is a tyrosine kinase inhibitor; for use in treatment of cancer.

An alternative of the second aspect of the invention relates to a combination medicament (and to methods of manufacture of such combination medicament) comprising or essentially consisting of
a. disulfiram; and
b. a second compound, wherein the second compound is a tyrosine kinase inhibitor; for use in treatment of cancer.

An alternative of the second aspect of the invention relates to a combination medicament (and to methods of manufacture of such combination medicament) comprising or essentially consisting of
a. dithiocarbamate; and
b. a second compound, wherein the second compound is a tyrosine kinase inhibitor; for use in treatment of cancer.

In certain embodiments, the second compound is selected from the group of a BRAF inhibitor, a panRAF inhibitor, a MEK inhibitor, and a dual RAF/MEK inhibitor.

In certain embodiments, the second compound is selected from the group of dabrafenib, encorafenib, vemurafenib, belvarafenib, selumetinib, binimetinib, cobimetinib, mirdametinib, pimasertib, selumetinib, trametinib, and avutometinib.

In certain embodiments, the second compound is a cKIT inhibitor. In certain embodiments, the second compound is selected from the group of imatinib, sunitinib and desatinib.

In certain embodiments, the cancer is selected from the group of melanoma, brain cancer, breast cancer, pancreatic cancer, lung cancer, and gastrointestinal cancer. In certain embodiments, the cancer is selected from the group of melanoma, medulloblastoma, glioblastoma, non-small cell lung cancer, and colon cancer. In certain embodiments, the cancer is melanoma. In certain embodiments, the cancer is metastatic melanoma.

### Medical Treatment

Similarly, within the scope of the present invention is a method or treating cancer in a patient in need thereof, comprising administering to the patient a compound according to the above description.

### Pharmaceutical Compositions, Administration/Dosage Forms and Salts

According to one aspect of the compound according to the invention, the compound according to the invention is provided as a pharmaceutical composition, pharmaceutical administration form, or pharmaceutical dosage form.

The skilled person is aware that any specifically mentioned drug compound mentioned herein may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

In certain embodiments of the invention, the compound of the present invention is typically formulated into pharmaceutical dosage forms to provide an easily controllable dosage of the drug and to give the patient an elegant and easily handleable product.

Similarly, a dosage form for the prevention or treatment of cancer is provided, comprising a non-agonist ligand or antisense molecule according to any of the above aspects or embodiments of the invention.

The invention further encompasses a pharmaceutical composition comprising a compound of the present invention, or a pharmaceutically acceptable salt thereof, and a pharmaceutically acceptable carrier. In further embodiments, the composition comprises at least two pharmaceutically acceptable carriers, such as those described herein.

Certain embodiments of the invention relate to a dosage form for enteral administration, such as nasal, buccal, rectal, transdermal or oral administration, or as an inhalation form or suppository. In addition, the pharmaceutical compositions of the present invention can be made up in a solid form (including without limitation capsules, tablets, pills, granules, powders or suppositories), or in a liquid form (including without limitation solutions, suspensions or emulsions).

Certain embodiments of the invention relate to a dosage form for parenteral administration, such as subcutaneous, intravenous, intrahepatic or intramuscular injection forms. Optionally, a pharmaceutically acceptable carrier and/or excipient may be present.

Certain embodiments of the invention relate to a dosage form for topical administration. The skilled artisan is aware of a broad range of possible recipes for providing topical formulations, as exemplified by the content of Benson and Watkinson (Eds.), Topical and Transdermal Drug Delivery: Principles and Practice (1st Edition, Wiley 2011, ISBN-13: 978-0470450291); and Guy and Handcraft: Transdermal Drug Delivery Systems: Revised and Expanded (2^{nd} Ed., CRC Press 2002, ISBN-13: 978-0824708610); Osborne and Amann (Eds.): Topical Drug Delivery Formulations (1^{st} Ed. CRC Press 1989; ISBN-13: 978-0824781835). In embodiments of the invention relating to topical uses of the compounds of the invention, the pharmaceutical composition is formulated in a way that is suitable for topical administration such as aqueous solutions, suspensions, ointments, creams, gels or sprayable formulations, e.g., for delivery by aerosol or the like, comprising the active ingredient together with one or more of solubilizers, stabilizers, tonicity enhancing agents, buffers and preservatives that are known to those skilled in the art.

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition or combination of the present invention can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

### Method of Manufacture and Method of Treatment According to the Invention

The invention further encompasses, as an additional aspect, the use of a compound as identified herein (neocuproine, elesclomol, disulfiram, or dithiocarbamate), or its pharmaceutically acceptable salt, as specified in detail above, for use in a method of manufacture of a medicament for the treatment or prevention of cancer.

Similarly, the invention encompasses methods of treatment of a patient having been diagnosed with a disease associated with cancer. This method entails administering to the patient an effective amount of a compound as identified herein (neocuproine, elesclomol, disulfiram, or dithiocarbamate), or its pharmaceutically acceptable salt, as specified in detail herein.

The invention further encompasses the following items:

### Items:

1. A compound selected from the group comprising neocuproine, elesclomol, disulfiram, and dithiocarbamate for use in treatment of cancer.
2. The compound for use according to item 1, wherein the compound is neocuproine.
3. The compound for use according to item 1, wherein the compound is elesclomol.
4. The compound for use according to item 1, wherein the compound is disulfiram.
5. The compound for use according to item 1, wherein the compound is dithiocarbamate.
6. The compound for use according to any one of the preceding items, wherein said cancer is characterized by cells having an NRAS mutation.
7. The compound for use according to item 6, wherein the NRAS mutation is selected from the group comprising an NRAS Q61 mutation, an NRAS G12 mutation and an NRAS G13 mutation.
8. The compound for use according to item 6, wherein the NRAS mutation is selected from the group comprising NRAS Q61R, NRAS Q61K, NRAS Q61L, and NRAS Q61H, NRAS G12D, NRAS G12S, NRAS G12D, NRAS G12C, NRAS G12V, NRAS G12A, NRAS G13D, NRAS G13R, NRAS G13V, NRAS G13C.
9. The compound for use according to item 6, wherein the NRAS mutation is selected from the group comprising NRAS Q61K, NRAS Q61L, NRAS Q61R, NRAS Q61H, or NRAS G12A.
10. The compound for use according to any one of the preceding items, wherein said cancer is characterized by cells having a BRAF mutation.
11. The compound for use according to item 10, wherein the BRAF mutation is selected from the group comprising BRAF V600E, BRAF V600K, BRAF V600D or BRAF V600R.
12. The compound for use according to any one of the preceding items, wherein said cancer is characterized by cells having a cKIT mutation.
13. The compound for use according to item 12, wherein the cKIT mutation is selected from the group comprising cKIT K642E, cKIT L576P, cKIT V559A and cKIT W557R.
14. The compound for use according to any one of the preceding items, wherein said cancer is characterized by cells having a KRAS mutation.
15. The compound for use according to item 14, wherein the KRAS mutation is selected from the group of a KRAS Q61 mutation, a KRAS G12 mutation, and a KRAS G13 mutation.
16. The compound for use according to item 14, wherein the KRAS mutation is selected from the group of KRAS Q61R, KRAS Q61K, KRAS Q61L, KRAS Q61H, KRAS G13D, KRAS G13R, KRAS G13V, KRAS G13C, KRAS G12D, KRAS G12S, KRAS G12D, KRAS G12C, KRAS G12V, and KRAS G12A.
17. The compound for use according to any one of the preceding items, wherein said cancer is characterized by cells having an HRAS mutation.
18. The compound for use according to item 17, wherein the HRAS mutation is selected from the group of an HRAS Q61 mutation, an HRAS G12 mutation, and an HRAS G13 mutation.
19. The compound for use according to item 17, wherein the HRAS mutation is selected from the group of HRAS Q61R, HRAS Q61K, HRAS Q61L, HRAS Q61H, HRAS G12D, HRAS G12S, HRAS G12D, HRAS G12C, HRAS G12V, HRAS G12A, HRAS G13D, HRAS G13R, HRAS G13V, and HRAS G13C.
20. The compound for use according to any one of the preceding items, wherein said cancer is characterized by cells characterized by a mutation in two genes of the group comprising NRAS as specified in item 6 to 9, BRAF as specified in item 10 and 11, and cKIT as specified in item 12 and 13.
21. The compound for use according to any one of the preceding items, wherein said cancer is characterized by cells characterized by a mutation in all three genes of the group comprising NRAS as specified in item 6 to 9, BRAF as specified in item 10 and 11, and cKIT as specified in item 12 and 13.
22. The compound for use according to any one of the preceding items, wherein said cancer is characterized by cells characterized by a mutation in two genes of the group NRAS, KRAS, HRAS, BRAF, cKIT, and NF1.
23. The compound for use according to any one of the preceding items, wherein said cancer is characterized by cells characterized by a mutation in three genes of the group NRAS, KRAS, HRAS, BRAF, cKIT, and NF1.
24. The compound for use according to any one of the preceding items, wherein said cancer is characterized by cells characterized by a mutation in four genes of the group NRAS, KRAS, HRAS, BRAF, cKIT, and NF1.
25. The compound for use according to any one of the preceding items, wherein said cancer is characterized by cells that are resistant or refractory to treatment with a MEK inhibitor, particularly a MEK inhibitor selected from trametinib, binimetinib, cobimetinib and selumetninib.
26. The compound for use according to any one of the preceding items, wherein said cancer is characterized by cells that are resistant to treatment with a BRAF inhibitor, particularly a BRAF inhibitor selected from vemurafenib, dabrafenib and encorafenib.
27. The compound for use according to any one of the preceding items, wherein said cancer is characterized by cells that are resistant to treatment with a cKIT inhibitor, particularly a cKIT inhibitor selected from imatinib, sunitinib, desatinimb or nilotinib.
28. The compound for use according to any one of the preceding items, wherein said cancer is characterized by cells
   a. having a BRAF mutation, and
   b. are resistant to treatment with a BRAF inhibitor.
29. The compound for use according to any one of the preceding items, wherein said cancer is characterized by cells
   a. having an NRAS mutation, and
   b. are resistant to treatment with a MEK inhibitor.
30. The compound for use according to any one of the preceding items, wherein said cancer is characterized by cells
   a. having a cKIT mutation, and
   b. are resistant to treatment with a cKIT inhibitor.
31. The compound for use according to any one of the preceding items, wherein said compound is co-administered in combination with a MAPK-inhibitor.
31 a) The compound for use according to item 31, wherein the compound is co-administered in combination with vemurafenib.
31 b) The compound for use according to item 31, wherein the compound is co-administered in combination with encorafenib.
31 c) The compound for use according to item 31, wherein the compound is co-administered in combination with dabrafenib.
31 d) The compound for use according to item 31, wherein the compound is co-administered in combination with trametinib.
31 e) The compound for use according to item 31, wherein the compound is co-administered in combination with binimetinib.
31 f) The compound for use according to item 31, wherein the compound is co-administered in combination with cobimetinib.
31 g) The compound for use according to item 31, wherein the compound is co-administered in combination with mirdametinib.
31 h) The compound for use according to item 31, wherein the compound is co-administered in combination with selumetinib.
31 i) The compound for use according to item 31, wherein the compound is co-administered in combination with imatinib.
31 j) The compound for use according to item 31, wherein the compound is co-administered in combination with sunitinib.
31 k) The compound for use according to item 31, wherein the compound is co-administered in combination with desatinib.
31 I) The compound for use according to item 31, wherein the compound is co-administered in combination with v nilotinib.
32. A combination medicament comprising or essentially consisting of
   a. neocuproine; and
   b. a compound selected from the group comprising vemurafenib, encorafenib, dabrafenib, trametinib, binimetinib, cobimetinib, mirdametinib, selumetinib, imatinib, sunitinib, desatinib and nilotinib;
   for use in treatment of cancer.
33. A combination medicament comprising or essentially consisting of
   a. elesclomol; and
   b. a compound selected from the group comprising vemurafenib, encorafenib, dabrafenib, trametinib, binimetinib, cobimetinib, mirdametinib, selumetinib, imatinib, sunitinib, desatinib and nilotinib;
   for use in treatment of cancer.
34. A combination medicament comprising or essentially consisting of
   a. disulfiram; and
   b. a compound selected from the group comprising vemurafenib, encorafenib, dabrafenib, trametinib, binimetinib, cobimetinib, mirdametinib, selumetinib, imatinib, sunitinib, desatinib and nilotinib;
   for use in treatment of cancer.
35. A combination medicament comprising or essentially consisting of
   a. dithiocarbamate; and
   b. a compound selected from the group comprising vemurafenib, encorafenib, dabrafenib, trametinib, binimetinib, cobimetinib, mirdametinib, selumetinib, imatinib, sunitinib, desatinib and nilotinib;
   for use in treatment of cancer.
36. A combination medicament comprising or essentially consisting of
   a. neocuproine; and
   b. a second compound, wherein the second compound is a MAPK-pathway inhibitor;
   for use in treatment of cancer.
37. The combination medicament for use according to item 36, wherein the second compound is selected from the group of a BRAF inhibitor, a panRAF inhibitor, a MEK inhibitor, and a dual RAF/MEK inhibitor.
38. The combination medicament for use according to item 36, wherein the second compound is a BRAF inhibitor.
39. The combination medicament for use according to item 36, wherein the second compound is a panRAF inhibitor.
40. The combination medicament for use according to item 36, wherein the second compound is a MEK inhibitor.
41. The combination medicament for use according to item 36, wherein the second compound is a dual RAF/MEK inhibitor.
42. The combination medicament for use according to item 36, wherein the second compound is selected from the group of dabrafenib, encorafenib, vemurafenib, belvarafenib, selumetinib, binimetinib, cobimetinib, mirdametinib, pimasertib, selumetinib, trametinib, and avutometinib.
43. A combination medicament comprising or essentially consisting of
   a. neocuproine; and
   b. a second compound, wherein the second compound is a tyrosine kinase inhibitor;
   for use in treatment of cancer.
44. The combination medicament for use according to item 43, wherein the second compound is selected from the group of imatinib, sunitinib and desatinib.
45. The compound or the combination medicament according to any one of the preceding items, wherein said cancer is selected from the group of melanoma, brain cancer, breast cancer, pancreatic cancer, lung cancer, and gastrointestinal cancer, particularly wherein said cancer is selected from the group of melanoma medulloblastoma, glioblastoma, non-small cell lung cancer, and colon cancer, more particularly wherein said cancer is melanoma, most particularly metastatic melanoma.

The invention is further illustrated by the following examples and figures, from which further embodiments and advantages can be drawn. These examples are meant to illustrate the invention but not to limit its scope.

### DESCRIPTION OF THE FIGURES

FIG. 1 shows chemical structures of ionophoric copper-chelators. A. Chemical structure of Neucuproine (supplier: TimTec, ST013881) used to create all experimental data in this application and is referring to the IUPAC name 2,9-Dimethyl-1,19-phenanthroline. B. Chemical structure of the compound Elesclomol C. Chemical structure of the compound Disulfiram. D. Sodium Diethyldithiocarbamate (DDC).

FIG. 2 shows the HPLC chromatogram for Neocuproine (Sigma-Aldrich) and ST013881 (TimTec). Samples of 10 mM stock solution of either ethanol for Neocuproine or DMSO for STO13881 were diluted 100× in 0.1% TFA and 1 I was injected three times (=0.1 nmol). Data were created under contract research condition by the functional genomics centre Zurich (FGCZ).

FIG. 3 shows growth inhibition curves for non-ionophoric copper chelators. (A) Melanoma cell cultures were treated with dose-escalating concentration up to 100 μM of trientine dihydrochloride. (B) Melanoma cell cultures were treated with dose-escalating concentrations up to 100 μM of tetrathiomolybdate. (C) IC50 values and C.I. (95%) for each individual cell culture treated with tetrathiomolybdate.

FIG. 4 shows growth inhibition can be rescued by the depletion of copper using the copper-chelator Trientine. Cell culture medium was incubated with Trientine and growth inhibition was performed with Neocuproine or Elesclomol (left panel) or Disulfiram or DDC (right panel)

FIG. 5 shows that Neocuproine, Elesclomol, Disulfiram and DDC effects can be rescued by an antioxidant in melanoma cell lines:
A. Viability after Neuocuproine treatment is rescued by the addition of an antioxidant NAC (N-acetyl-Lcystein). Four different melanoma cultures harbouring either NRAS mutation (M130219, M130227, M160915) or a double NRAS/BRAF mutation (M121224) were treated with 250 nM or 500 nM of Neuocuproine alone or in combination with 1 mM of NAC for 72h. Viability was measured with a standard Resazaurin-based viability assay and % of viable cells were calculated in relation to vehicle treated cells.
B. Viability after Elesclomol treatment is rescued by the addition of an antioxidant NAC (N-acetyl-Lcystein). Four different melanoma cultures harbouring either NRAS mutation (M130219, M130227, M160915) or a double NRAS/BRAF mutation (M121224) were treated with 250 nM or 500 nM of Neuocuproine alone or in combination with 1 mM of NAC for 72h. Viability was measured with a standard Resazaurin-based viability assay and % of viable cells were calculated in relation to vehicle treated cells.
C. Viability after Disulfiram treatment is rescued by the addition of an antioxidant NAC (N-acetyl-Lcystein). Four different melanoma cultures harbouring either NRAS mutation (M130219, M130227, M160915) or a double NRAS/BRAF mutation (M121224) were treated with 250 nM or 500 nM of Neuocuproine alone or in combination with 1 mM of NAC for 72h. Viability was measured with a standard Resazaurin-based viability assay and % of viable cells were calculated in relation to vehicle treated cells.
D. Viability after DDC treatment is rescued by the addition of an antioxidant NAC (N-acetyl-Lcystein). Four different melanoma cultures harbouring either NRAS mutation (M130219, M130227, M160915) or a double NRAS/BRAF mutation (M121224) were treated with 250 nM or 500 nM of Neuocuproine alone or in combination with 1 mM of NAC for 72h. Viability was measured with a standard Resazaurin-based viability assay and % of viable cells were calculated in relation to vehicle treated cells.

FIG. 6 shows that Neocuproine, Elesclomol, Disulfiram and DDC treatment is inducing apoptosis in melanoma cell lines dependent on an oxidative effect
A. Induction of apoptosis is measured in melanoma cell lines after treatment with Neocuproine (1 M, 48h) in the presence or absence of NAC. Induction of apoptosis if determined by Caspase 3/7 activity in four different melanoma cultures harbouring either NRAS mutation (M130219, M130227, M160915) or a double NRAS/BRAF mutation (M121224). Measurements are normalized to control experiments treated with the vehicle DMSO. The bar graphs represent the average of independent experiments and show that Neocuproine is inducing caspase-dependent apoptosis in all melanoma cell lines and that the effect is rescued by the antioxidant NAC.
B. Induction of apoptosis is measured in melanoma cell lines after treatment with Elesclomol (100 nM, 48h) in the presence or absence of NAC. Induction of apoptosis if determined by Caspase 3/7 activity in four different melanoma cultures harbouring either NRAS mutation (M130219, M130227, M160915) or a double NRAS/BRAF mutation (M121224). Measurements are normalized to control experiments treated with the vehicle DMSO. The bar graphs represent the average of independent experiments and show that Elesclomol is inducing caspase-dependent apoptosis in all melanoma cell lines and that the effect is rescued by the antioxidant NAC.
C. Induction of apoptosis is measured in melanoma cell lines after treatment with Elesclomol (100 nM, 48h) in the presence or absence of NAC. Induction of apoptosis if determined by Caspase 3/7 activity in three different melanoma cultures harbouring either NRAS mutation (M130219, M130429) or a double NRAS/BRAF mutation (M121224). Measurements are normalized to control experiments treated with the vehicle DMSO. The bar graphs represent the average of independent experiments and show that Elesclomol is inducing caspase-dependent apoptosis in all melanoma cell lines and that the effect is rescued by the antioxidant NAC.
D. Induction of apoptosis is measured in melanoma cell lines after treatment with Disulfiram (1 M, 48h) in the presence or absence of NAC. Induction of apoptosis if determined by Caspase 3/7 activity in three different melanoma cultures harbouring either NRAS mutation (M130219, M130227) or a double NRAS/BRAF mutation (M121224). Measurements are normalized to control experiments treated with the vehicle DMSO. The bar graphs represent the average of independent experiments and show that Disulfiram is inducing caspase-dependent apoptosis in all melanoma cell lines and that the effect is rescued by the antioxidant NAC.
E. Induction of apoptosis is measured in melanoma cell lines after treatment with DDC (1 M, 48h) in the presence or absence of NAC. Induction of apoptosis if determined by Caspase 3/7 activity in three different melanoma cultures harbouring either NRAS mutation (M130219, M130227) or a double NRAS/BRAF mutation (M121224). Measurements are normalized to control experiments treated with the vehicle DMSO. The bar graphs represent the average of independent experiments and show that Disulfiram is inducing caspase-dependent apoptosis in all melanoma cell lines and that the effect is rescued by the antioxidant NAC.

FIG. 7 A. Inhibition of cell invasion of 3D spheroids into a Collagen Type I matrix by Neocuproine, Elesclomol, Disulfiram and DDC.
Melanoma cell line M160915 were grown as 3D spheroids and embedded into a Collagen Type I matrix. Spheroids were treated for 5 days either with Neocuproine (1 M), Elesclomol (100 nM), Disulfiram (1 M) or DDC (1 M) alone or in combination with the MEK inhibitors Binimetinib (500 nM) or Mirdametinib (500 nM). Viable cells were stained with the live-cell dye Calcein-Green-AM (green staining) while dead cells were stained with Ethidium Homodimers (red staining) and live-imaging was performed using a fluorescent microscope. (A) One representative fluorescent image for each condition is shown in this panel. (Scale bar 250 m). (B) The % area of invasion” was determined using ImageJ software for each condition. Multiple images per condition (total of 4 spheres/condition) were analysed and bar graphs were plotted using GraphPad Prism software.
C. Melanoma cell line M121224 was grown as 3D spheroids and embedded into a Collagen Type I matrix. Spheroids were treated for 5 days either with Neocuproine (1 M), Elesclomol (100 nM), Disulfiram (1 M) or DDC (1 M) alone or in combination with the MAPK inhibitors Encorafenib, Binimetinib (500 nM) or Mirdametinib (500 nM). Viable cells were stained with the live-cell dye Calcein-Green-AM (green staining) while dead cells were stained with Ethidium Homodimers (red staining) and live-imaging was performed using a fluorescent microscope. One representative fluorescent image for each condition is shown in this panel. (Scale bar 250 m).
(D) The % area of invasion” was determined using ImageJ software for each condition. Multiple images per condition (total of 4 spheres/condition) were analysed and bar graphs were plotted using GraphPad Prism software.

FIG. 8 In vivo efficacy. Immunocompromise mice were injected into the flank with cell line M130227. Mice were treated with vehicle, MEK inhibitor (MEKi, Binimetinib=MEK162), or the combination of MEKi and the indicated compound when first palpable tumors were visible.
A. (upper panel) Summarized tumor volumes of individual treatment groups (vehicle, MEKi, Neocuproine+MEKi)). (lower panel) Kaplan-Meier survival analysis processed with Graph Pad Prism software for the combination of Neuocuproine+MEKi
B. Summarized tumor volumes of individual treatment groups (vehicle, MEKi, Elesclomol+MEKi)).
C. Summarized tumor volumes of individual treatment groups (vehicle, MEKi, Disulfiram+MEKi)).

FIG. 9 In vitro viability assay for the comparison of viability reduction of neocuproine monotherapy, MAPK-pathway inhibitor monotherapy and the neocuproine+MAPK-pathway inhibitor combination at 72h of treatment, at the indicated concentrations in NRAS mutated cancer cells. Viabilities are normalized to the untreated DMSO vehicle control (NEO 0 nM). Depicted are Neocuproine monotherapy (NEO), The MAPK-pathway inhibitor only treatment (NEO+depicted MAPK-pathway inhibitor at NEO 0 nM), and the combination with MAPK-pathway inhibitors at the indicated concentration (NEO+depicted MAPK-pathway inhibitor). A: M010817 (Melanoma, NRAS Q61R); B: ONS76 (Brain Cancer, NRAS Q61R)

FIG. 10 In vitro viability assay for the comparison of viability reduction of neocuproine monotherapy, MAPK-pathway inhibitor monotherapy and the neocuproine+MAPK-pathway inhibitor combination at 72h of treatment, at the indicated concentrations in KRAS mutated cancer cells. Viabilities are normalized to the untreated DMSO vehicle control (NEO 0 nM). Depicted are Neocuproine monotherapy (NEO), The MAPK-pathway inhibitor only treatment (NEO+depicted MAPK-pathway inhibitor at NEO 0 nM), and the combination with MAPK-pathway inhibitors at the indicated concentration (NEO+depicted MAPK-pathway inhibitor).
A. LS180 (Gastro-intestinal cancer, KRAS G12S)
B. A549 (Lung cancer, KRAS G12S)
C. CAPAN-2 (Pancreatic cancer, KRAS G12V)
D. MIA PaCa-2 (Pancreatic cancer, G12C)
E. MDA-MB-231 (Breast cancer, KRAS G13D)

FIG. 11 In vitro viability assay for the comparison of viability reduction of neocuproine monotherapy, MAPK-pathway inhibitor monotherapy and the neocuproine+MAPK-pathway inhibitor combination at 72h of treatment, at the indicated concentrations in BRAF mutated cancer cells. Viabilities are normalized to the untreated DMSO vehicle control (NEO 0 nM). Depicted are Neocuproine monotherapy (NEO), The MAPK-pathway inhibitor only treatment (NEO+depicted MAPK-pathway inhibitor at NEO 0 nM), and the combination with MAPK-pathway inhibitors at the indicated concentration (NEO+depicted MAPK-pathway inhibitor).
A. M00921 (Melanoma, BRAF V600E)
B. COLO 205 (Gastro-intestinal cancer, BRAF V600E)

FIG. 12 In vitro viability assay for the comparison of viability reduction of neocuproine monotherapy, MAPK-pathway inhibitor monotherapy and the neocuproine+MAPK-pathway inhibitor combination at 72h of treatment, at the indicated concentrations in NF1 mutated cancer cells. Viabilities are normalized to the untreated DMSO vehicle control (NEO 0 nM). Depicted are Neocuproine monotherapy (NEO), The MAPK-pathway inhibitor only treatment (NEO+depicted MAPK-pathway inhibitor at NEO 0 nM), and the combination with MAPK-pathway inhibitors at the indicated concentration (NEO+depicted MAPK-pathway inhibitor).
NCHI-H1838 (Lung cancer, NF1 loss)

FIG. 13 In vitro viability assay for the comparison of viability reduction of neocuproine monotherapy, MAPK-pathway inhibitor or tyrosine kinase inhibitor monotherapy and the neocuproine+MAPK-pathway inhibitor or the neocuproine+tyrosine kinase inhibitor combination at 72h of treatment, at the indicated concentrations in cKIT mutated cancer cells. Viabilities are normalized to the untreated DMSO vehicle control (NEO 0 nM). Depicted are Neocuproine monotherapy (NEO), the MAPK-pathway inhibitor only or tyrosine kinase inhibitor only treatment (NEO+depicted MAPK-pathway inhibitor or tyrosine kinase inhibitor at NEO 0 nM), and the combination with MAPK-pathway inhibitors or tyrosine kinase inhibitors at the indicated concentration (NEO+depicted MAPK-pathway inhibitor or NEO+depicted tyrosine kinase inhibitor).
A: M100513 (Melanoma, cKIT K642E)
B: M150207 (Melanoma, cKIT K642E)

### EXAMPLES

### Example 1: Neocuproine, Elesclomol, Disulfiram and DDC are Inhibiting the Growth of Primary Melanoma Cancer Cells

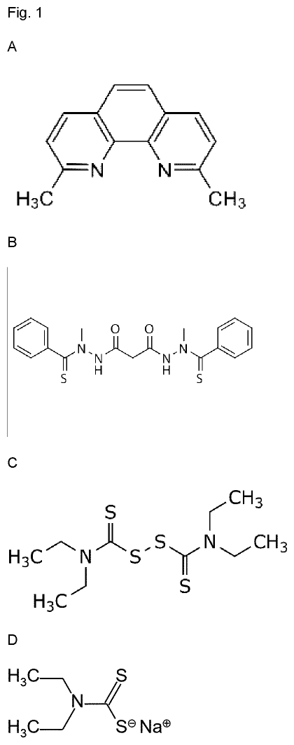

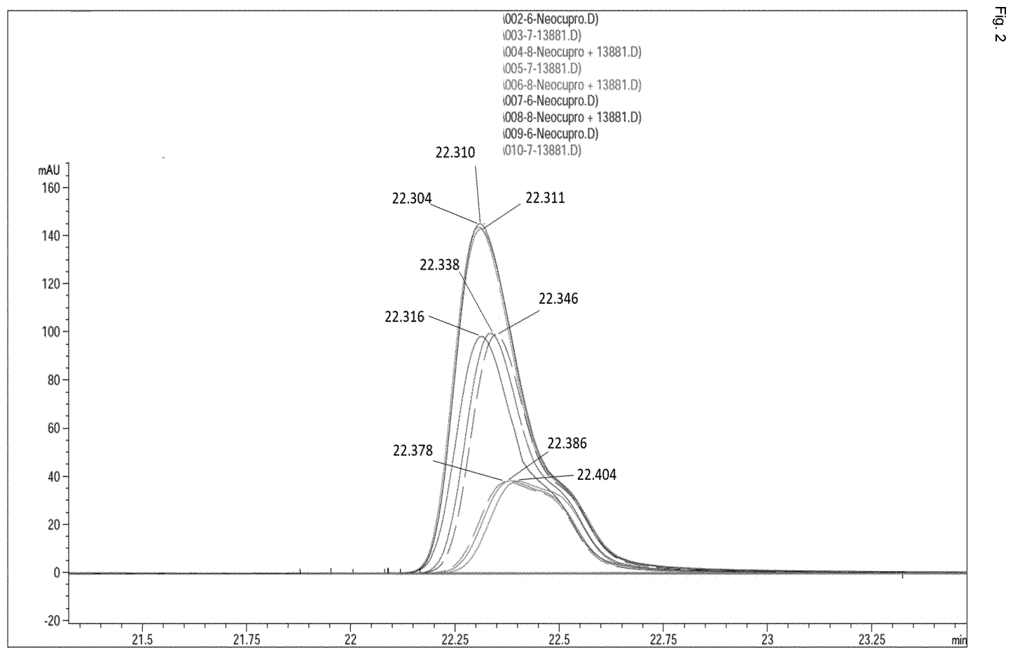

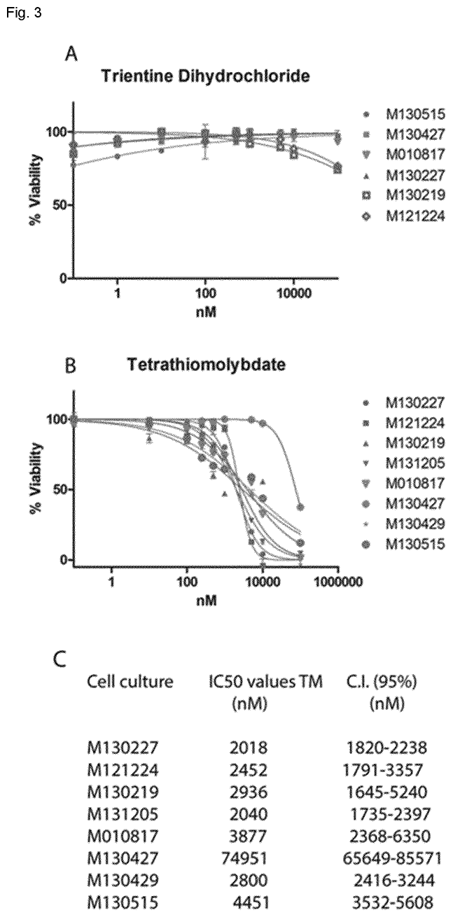

In order to find compounds which can target MAPK activated cancer cells, including those which are resistant to MAPK inhibitors, we used melanoma as a model. Melanoma are known to be driven mainly though activations in the MAPK pathway. Indeed 70-80% of all melanomas harbour an activating mutation in the RAS or RAF oncogene. We performed a drug screen with so called “small molecule compounds” on MAPK-pathway inhibitor treatment-resistant and primary patient-derived melanoma cells. For this approach, the inventors chose to screen cell viability against the ActiTarg-K Library from TimTec, which is composed of 960 small molecules with potential kinase inhibitory function. The inventors found compound STO13881 (Neocuproine) to be a potent inhibitor of proliferation on primary melanoma cell cultures harbouring NRAS, BRAF, NRAS/BRAF double mutation or cKit mutations (chemical structure see FIG. 1A). Additionally, we also performed viability assays with other ionophoric copper chelators (Disulfiram, Elesclomol) on other cancer cells derived from solid tumors with activating MAPK status (Example 10-13).

Patient-derived primary melanoma cells were grown from surplus tumour material after surgery. Patient material including cell cultures are embedded into a biobank after signed consent. Cell culture mutation status for the most common mutations in melanoma (e.g. NRAS, BRAF, cKit, PTEN) is confirmed routinely as well as the mutation status of the corresponding tumour material.

The IC50 concentration (inhibitory concentration at which 50% cell viability is observed) of the compound for each individual cell culture was determined as follow: Briefly, cells were seeded into 96-well plates (seeding densities see experimental table 2) in 90 μl of appropriate cell culture medium (according to experimental table 2) and let to adhere overnight. Cell seeding density was pre-tested and differs between cell cultures according to their individual proliferation rate. The seeding density was selected to give rise to 80% cell confluence at the last day of the experiment (72h time point). Neocuproine (STO13881, TimTec, Cat Nr. MFCD00004973; CAS: 484-11-7) was diluted in DMSO (dimethylsufoxide, CAS No. 67-68-5) to a 1000-fold higher stock solution than the final end concentration in the culture wells starting with 10 mM, using a serial dilution schedule. The compound was then further diluted 1:100 in cell culture medium in a separate 96-well plate. From this 1:100 dilution plate, 10 μl were transferred to the 96-well plate containing the cancer cells using a multichannel pipet, resulting in the desired 1:1000 final concentration from the 1000-fold higher stock solution. The final concentrations were 1 nM, 10 nM, 100 nM, 250 nM, 500 nM, 1000 nM, 5000 nM and 10000 nM. The solvent (here DMSO) served as control and was added in the appropriate concentration to the control wells. After 72h cell viability was measured with a standard viability assay using resazurin (7-hydroxy-3H-phenoxazin-3-one 10-oxide, CAS No. 550-82-3, 0.15 mg/ml in PBS). Per 96-well culture plate 1 ml of resazurin solution was mixed with 9 ml cell culture medium. The medium of the melanoma cell containing 96-well plate was exchanged with 100 μl/well resazurin/medium mixture. Plates were then incubated in the tissue culture incubator for 2-4 h at 37° C./5% CO₂ and fluorescent intensity was measured in a standard fluorescence plate reader (Tecan) with the following parameter: Excitation 535 nm/Emission 595 nm. Data are analysed by GraphPad Prism Software. DMSO treated wells are set to 100% viability and IC50 values are calculated accordingly (see summary of IC50 data, experimental table 3).

Experimental table 1: List of cell culture media used

| Volume | | Cell culture | Cell culture | Cell culture | Cell culture medium |
|---|---|---|---|---|---|
| [mL] | | medium 1 | medium 2 | medium 3 | 4 |
| 500 | ml | RPMI1640 | DMEM (Cat.No. | McCoy's 5a | RPMI1640 (Sigma- |
| | | (Sigma-Aldrich) | 41966-029, Gibco) | Medium Modified | Aldrich) |
| | | | | (Cat.No. 30-2007, | |
| | | | | ATCC) | |
| 50 | ml | Heat-inactivated | Heat-inactivated | Heat-inactivated | Heat-inactivated FBS |
| | | FBS (Cat.No. P30- | FBS (Cat.No. | FBS (Cat.No. | (Cat.No. P30-1902, |
| | | 1902, PAN | P30-1902, PAN | P30-1902, PAN | PAN Biotech) |
| | | Biotech) | Biotech) | Biotech) | |
| 10 | ml | L-Glutamine, | | | L-Glutamine, 200 mM |
| | | 200 mM (Gibco) | | | (Gibco) |
| 5 | ml | Sodium pyruvate, | | | Sodium pyruvate, |
| | | 100 mM (Sigma- | | | 100 mM (Sigma- |
| | | Aldrich) | | | Aldrich) |
| | | | | | SCF 50 ng/ml final |
| | | | | | concentration |
| | | | | | (Cat.No. PHC2111, |
| | | | | | Fisher) |
| | | Vacuum-filtration, | Vacuum-filtration, | Vacuum-filtration, | Vacuum-filtration, |
| | | 0.22 μM pore size | 0.22 μM pore size | 0.22 μM pore size | 0.22 μM pore size |
| | | (TPP) | (TPP) | (TPP) | (TPP) |

Experimental table 1: Seeding-densities 96-well plate (for proliferation assay)

| Cell culture | Medium | Density*** | Cancer | Mutation |
|---|---|---|---|---|
| MM151001 | 1 | 2500 | Melanoma | NRAS Q61R |
| M130219 | 1 | 2000 | Melanoma | NRAS Q61R |
| M050829 | 1 | 3000 | Melanoma | NRAS Q61K |
| M130227 | 1 | 2000 | Melanoma | NRAS Q61R |
| MM070221 | 1 | 2500 | Melanoma | NRAS Q61R |
| M130926 | 1 | 2500 | Melanoma | NRAS Q61R |
| M131205 | 1 | 2500 | Melanoma | NRAS Q61R |
| M160915 | 1 | 2500 | Melanoma | NRAS Q61R |
| M130405 | 1 | 2500 | Melanoma | NRAS Q61R |
| M130429 | 1 | 2500 | Melanoma | NRAS Q61R |
| M130830 | 1 | 2500 | Melanoma | NRAS Q61K |
| M130515 | 1 | 2500 | Melanoma | NRAS Q61R |
| M130425 | 1 | 2500 | Melanoma | NRAS Q61R |
| M130427 | 1 | 2500 | Melanoma | NRAS Q61R |
| M010817 | 1 | 3500 | Melanoma | NRAS Q61R |
| M121224 | 1 | 2000 | Melanoma | BRAF V600E/NRAS Q61K |
| M140307 | 1 | 2500 | Melanoma | BRAF V600E/NRAS G12A |
| M150423 | 1 | 3000 | Melanoma | BRAF V600E/NRAS Q61R |
| M130903 | 1 | 2500 | Melanoma | BRAF V600E/NRAS Q61H |
| M170917 | 1 | 2500 | Melanoma | BRAF V600E/NRAS Q61R |
| M150506 | 1 | 2000 | Melanoma | BRAF V600E |
| M150672 | 1 | 2000 | Melanoma | BRAF V600E |
| M150619 | 1 | 2000 | Melanoma | BRAF V600E |
| M111031 | 1 | 3500 | Melanoma | BRAF V600E |
| M130820 | 1 | 2500 | Melanoma | BRAF V600E |
| M000921 | 1 | 1500 | Melanoma | BRAF V600E |
| M980513 | 1 | 3000 | Melanoma | BRAF V600E |
| M150536 | 1 | 2000 | Melanoma | BRAF V600E |
| M140625 | 1 | 2000 | Melanoma | BRAF V600E |
| M150548 | 1 | 2000 | Melanoma | BRAF V600E |
| M100511 | 4 | 3000 | Melanoma | cKit K642E |
| M100512 | 4 | 3000 | Melanoma | cKit K642E |
| M100513 | 4 | 3000 | Melanoma | cKit K42E |
| M100916 | 4 | 3500 | Melanoma | cKit K642E |
| M150207 | 4 | 3000 | Melanoma | cKit K642E |
| M150218 | 4 | 5000 | Melanoma | cKit K642E |
| M140904 | 4 | 2000 | Melanoma | cKit K642E |
| ONS76 | 1 | 2500 | Brain cancer | NRAS Q61R |
| NCIH1838 | 1 | 5000 | Lung cancer | NF1loss |
| MDA-MB-231 | 2 | 3500 | Breast Cancer | KRAS G13D |
| CAPAN2 | 3 | 3000 | Pancreas | KRAS G12V |
| A549 | 2 | 3000 | Lung cancer | KRAS G12S |
| LS180 | 2 | 2500 | Gastrointestinal | KRAS G12D |
| | | | cancer | |
| MIAPACA2 | 2 | 3000 | Pancreas | KRAS G12C |
| Colo 205 | 1 | 2000 | Gastrointestinal | BRAF V600E |
| | | | cancer | |
| NHD | 1 | 1500 | Non-cancer | |
| fibroblasts | | | | |
| Melanocytes | * | 2500 | Non-cancer | |
| Keratinocytes | ** | 3000 | Non-cancer | |

* Melanocyte cell culture medium: Melanocyte Growth Medium (Ready-to-use) - Includes Basal Medium and Supplement Mix (Cat. No. C-24010, PromoCell)
** Keratinocyte cell culture medium: KBM ™ Gold Keratinocyte Growth Basal Medium (Cat.No. 00192151, Lonza)
*** Seeding density (96well plate) (cells/well)

All IC50 values for Neocuproine were summarized in experimental table 3.

Experimental table 3: Overview of primary cell cultures used to create data in this application. Non-cancer cell cultures derived from humans did not show any cytotoxicity against Neocuproine. Detailed information of primary melanoma cell cultures with annotation of specific NRAS, BRAF or cKit mutation and the IC50 values (in nM) of Neocuproine. The IC50 is the accumulation of independent experiments where cells have been challenged with dose-escalating concentration of the compound. IC50 value were calculated using GraphPad Prism software.

| | | Neocuproine |
|---|---|---|
| Cell culture | Mutation | IC50 values (nM) |
| MM151001 | NRAS Q61R | 60 |
| M130219 | NRAS Q61R | 200 |
| M050829 | NRAS Q61K | 180 |
| M130227 | NRAS Q61R | 180 |
| MM070221 | NRAS Q61R | 1190 |
| M130926 | NRAS Q61R | 1200 |
| M131205 | NRAS Q61R | 570 |
| M160915 | NRAS Q61R | 400 |
| M130405 | NRAS Q61R | 4500 |
| M130429 | NRAS Q61R | <10000 |
| M130830 | NRAS Q61K | 1000 |
| M130515 | NRAS Q61R | 1000 |
| M130425 | NRAS Q61R | 3200 |
| M130427 | NRAS Q61R | 1700 |
| M010817 | NRAS Q61R | 1300 |
| M121224 | BRAF V600E/NRAS Q61K | 330 |
| M140307 | BRAF V600E/NRAS G12A | 200 |
| M150423 | BRAF V600E/NRAS Q61R | <10000 |
| M130903 | BRAF V600E/NRAS Q61H | 240 |
| M170917 | BRAF V600E/NRAS Q61R | 160 |
| M150506 | BRAF V600E | 530 |
| M150672 | BRAF V600E | 2700 |
| M150619 | BRAF V600E | 200 |
| M111031 | BRAF V600E | 950 |
| M130820 | BRAF V600E | 1560 |
| M000921 | BRAF V600E | 160 |
| M980513 | BRAF V600E | 330 |
| M150536 | BRAF V600E | 60 |
| M140625 | BRAF V600E | 220 |
| M150548 | BRAF V600E | 77 |
| M100511 | cKit 642E | 55 |
| M100512 | cKit 642E | 108 |
| M100513 | cKit 642E | 106 |
| M100916 | cKit 642E | 53 |
| M150207 | cKit 642E | 182 |
| M150218 | cKit 642E | 312 |
| M140904 | cKit 642E | 312 |
| ONS76 | NRAS Q61R | 179 |
| NCIH1838 | NF1loss | 747 |
| MDA-MB-231 | KRAS G13D | 604 |
| CAPAN2 | KRAS G12V | 698 |
| A549 | KRAS G12S | 203 |
| LS180 | KRAS G12D | 416 |
| MIAPACA2 | KRAS G12C | 568 |
| Colo 205 | BRAF V600E | 183 |

Following this finding, we evaluated other ionophoric copper-chelators and found Elesclomol (FIG. 1B, Experimental table 4), Disulfiram (FIG. 1C, Experimental table 5) and DDC (FIG. 1D, Experimental table 6) to also target melanoma cell lines with the oncogenic mutation in NRAS, BRAF or cKit. Experiments were conducted as described above.

Experimental table 4: Viability after DDC treatment is rescued by the addition of an antioxidant NAC (N-acetyl-Lcystein). Four different melanoma cultures harbouring either NRAS mutation (M130219, M130227, M160915) or a double NRAS/BRAF mutation (M121224) were treated with 250 nM or 500 nM of Neuocuproine alone or in combination with 1 mM of NAC for 72h. Viability was measured with a standard Resazaurin-based viability assay and % of viable cells were calculated in relation to vehicle treated cells.

| | | Elesclomol |
|---|---|---|
| Cell culture | Mutation | IC50 values (nM) |
| M130219 | NRAS(Q61R) | 6 |
| M130227 | NRAS(Q61R) | 11 |
| M160915 | NRAS(Q61R) | 193 |
| M130429 | NRAS(Q61R) | <10000 |
| M010817 | NRAS(Q61R) | 300 |
| M121224 | BRAF(V600E)/NRAS(Q61K) | 26 |
| M150672 | BRAF(V600E) | 240 |
| M000921 | BRAF(V600E) | 28 |
| M980513 | BRAF(V600E) | 45 |
| M100513 | cKit (642E) | 16 |
| M100916 | cKit (642E) | 16 |
| M150207 | cKit (642E) | 22 |
| M140904 | cKit (642E) | 12 |

Experimental table 5: Overview of primary cell cultures used to create data in this application. Detailed information of primary melanoma cell cultures with annotation of specific NRAS, BRAF or cKit mutation and the IC50 values of Disulfiram. The IC50 is the accumulation of independent experiments where cells have been challenged with dose-escalating concentration of the compound. IC50 value were calculated using GraphPad Prism software.

| | | Disulfiram |
|---|---|---|
| Cell culture | Mutation | IC50 values (nM) |
| M130219 | NRAS(Q61R) | 48 |
| M130227 | NRAS(Q61R) | 34 |
| M130429 | NRAS(Q61R) | <10000 |
| M010817 | NRAS(Q61R) | 51 |
| M121224 | BRAF(V600E)/NRAS(Q61K) | 53 |
| M150672 | BRAF(V600E) | 987 |
| M000921 | BRAF(V600E) | 30 |
| M980513 | BRAF(V600E) | 116 |
| M100916 | cKit (642E) | 60 |
| M150207 | cKit (642E) | 61 |

Experimental table 6: Overview of primary cell cultures used to create data in this application. Detailed information of primary melanoma cell cultures with annotation of specific NRAS, BRAF or cKit mutation and the IC50 values of Overview of primary cell cultures used to create data in this application. Detailed information of primary melanoma cell cultures with annotation of specific NRAS, BRAF or cKit mutation and the IC50 values of Disulfiram. The IC50 is the accumulation of independent experiments where cells have been challenged with dose-escalating concentration of the compound. IC50 value were calculated using GraphPad Prism software. Also shown here are the confident intervals when calculation was possible (95% certainty of IC50 value laying in this range). The IC50 is the accumulation of independent experiments where cells have been challenged with dose-escalating concentration of the compound. IC50 value were calculated using GraphPad Prism software.

| Cell | | DDC |
|---|---|---|
| culture | Mutation | IC50 values (nM) |
| M130219 | NRAS(Q61R) | 198 |
| M130227 | NRAS(Q61R) | 103 |
| M130429 | NRAS(Q61R) | <10000 |
| M010817 | NRAS(Q61R) | 137 |
| M121224 | BRAF(V600E)/NRAS(Q61K) | 142 |
| M150672 | BRAF(V600E) | 2450 |
| M000921 | BRAF(V600E) | 6 |
| M980513 | BRAF(V600E) | 37 |
| M100916 | cKit (642E) | 1920 |
| M150207 | cKit (642E) | 805 |

### Example 2: STO13881 has the IUPAC Name 2,9-dimethylpyridino[3,2-h]quinolone and has Overlapping HPLC Profiling to Neocuproine

STO13881 was referred to 2,9-dimethyl-1,10-phenanthrolin (neocuproine, CAS No. 484-11-7). High performance liquid chromatography (HPLC) analysis of compound STO13881 (TimTec) and neocuproine (Sigma-Aldrich) was performed (FIG. 2).

Although there is a very slight shift in the retention time between the two samples, they co-elute when injected together (see FIG. 2). It was concluded that STO13881 is neocuproine.

Data are created under contract research with the functional genomic center Zurich (FGCZ).

### Example 3: The Effect of Other Copper Chelators on Treatment of Resistant Melanoma Cell Cultures

There are two other copper chelators suggested for the treatment of cancer, namely trientine and tetrathiomolybdate (TTM). In contrast to Neocouproine or Elesclomol they are not suggested to be ionophoric. Their mode of action was described to decrease Copper availability for the cells. Especially in melanoma, Copper is a cofactor for MEK1.

The inventors purchased both substances from Sigma-Aldrich (trientine hydrochloride, Cat. Nr. 38260-01-4 and tetrathiomolybdate Cat. Nr. 323446) and tested them in a proliferation assay on a representative subset of melanoma cell cultures listed in table 3. Viability assay was conducted as described in Example 1: Neocuproine is inhibiting the growth of primary melanoma cancer cells. Final drug concentrations were 1 nM, 10 nM, 100 nM, 500 nM, 1000 nM, 5000 nM, 10000 nM and 100000 nM.

Trientine which is used to treat patients with Wilson's disease did not show any growth inhibitory effects on melanoma cell culture. The in vivo anti-tumor effect described by Brady et al. might be due to clearance of copper from the blood rather than effecting melanoma cells directly.

The copper chelator TTM showed growth inhibitory effects with, except for one example (IC50=74951 nM), IC50 values ranging from 2018 nM to 4451 nM, These IC50 values are considerably higher compared to the IC 50 values of STO13881 (experimental table 3).

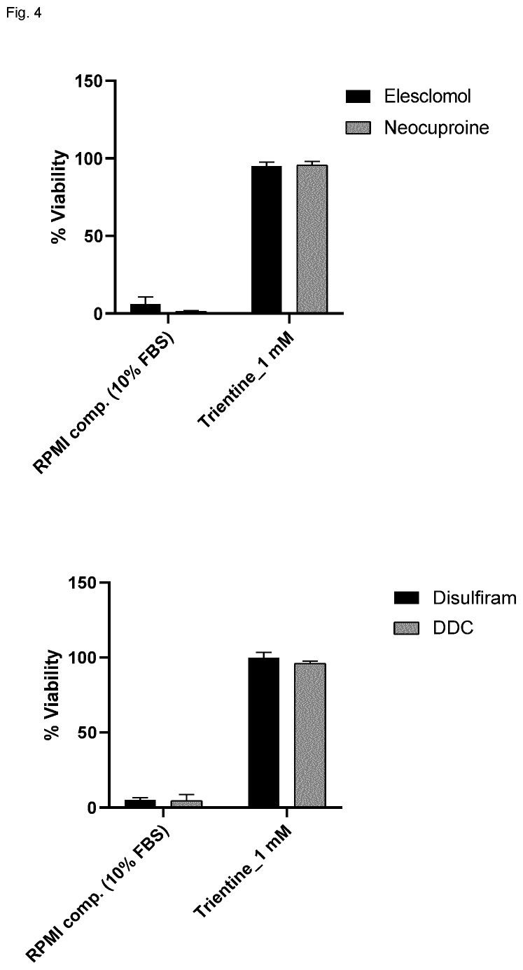

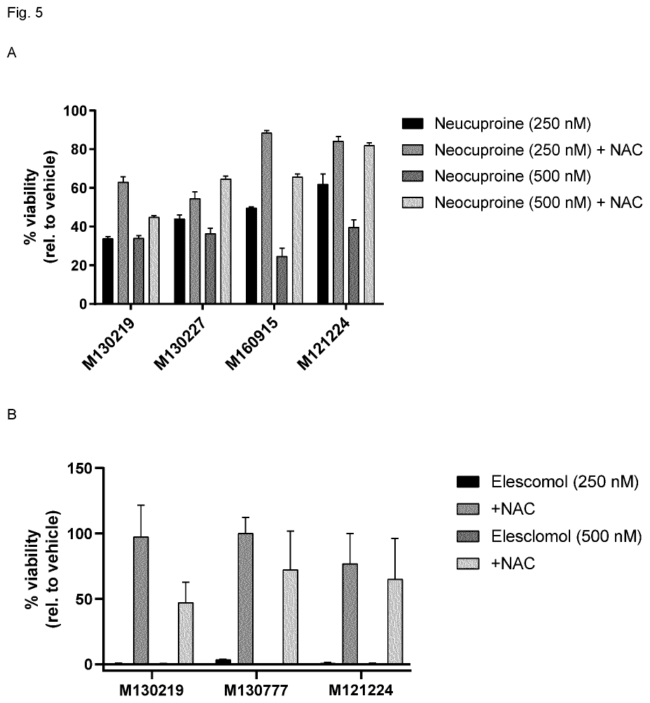

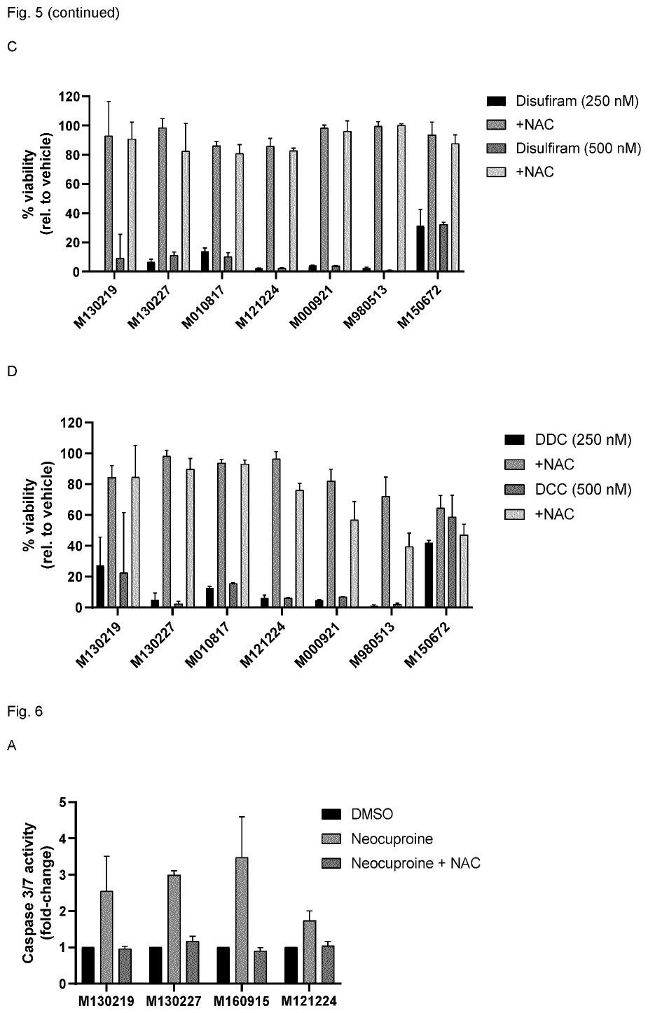

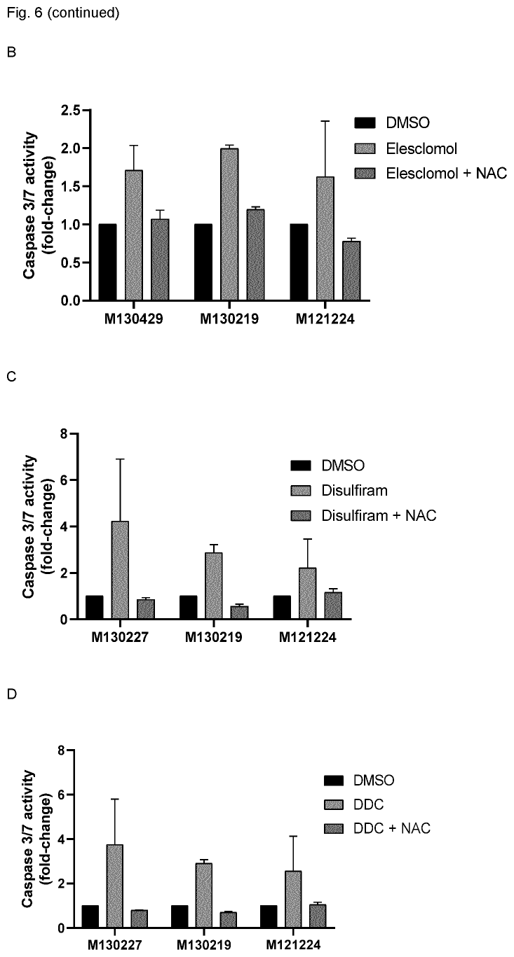

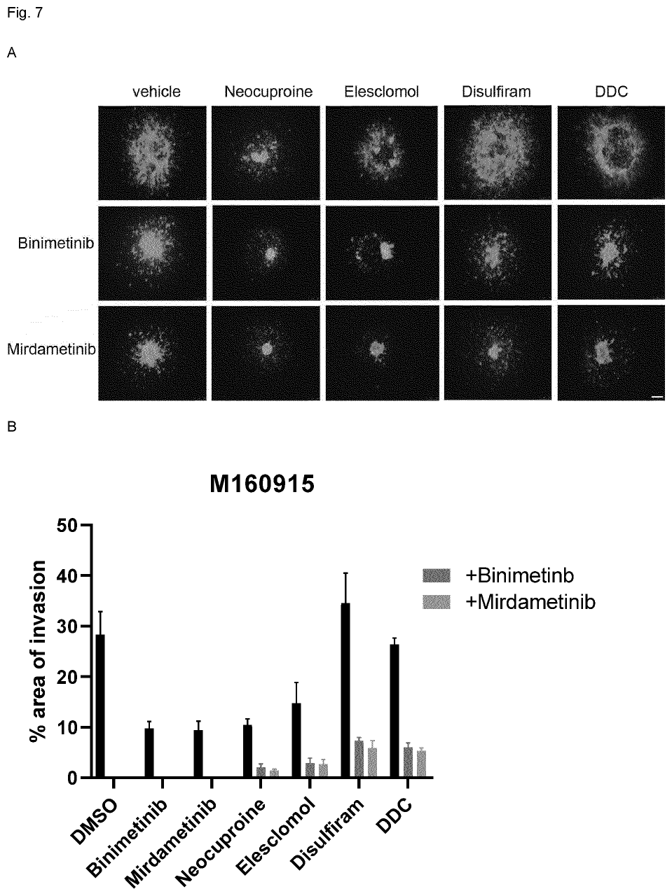

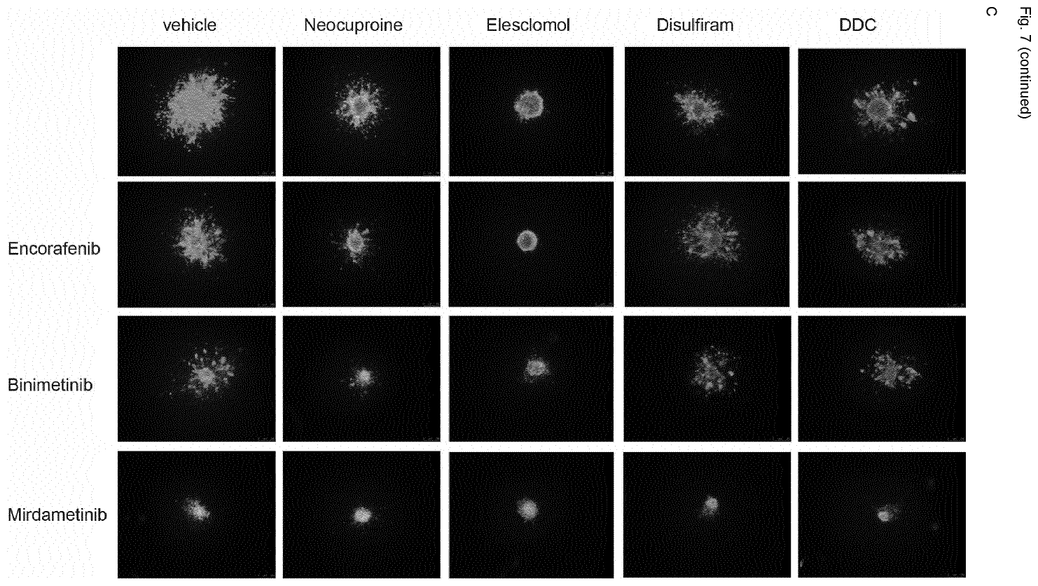

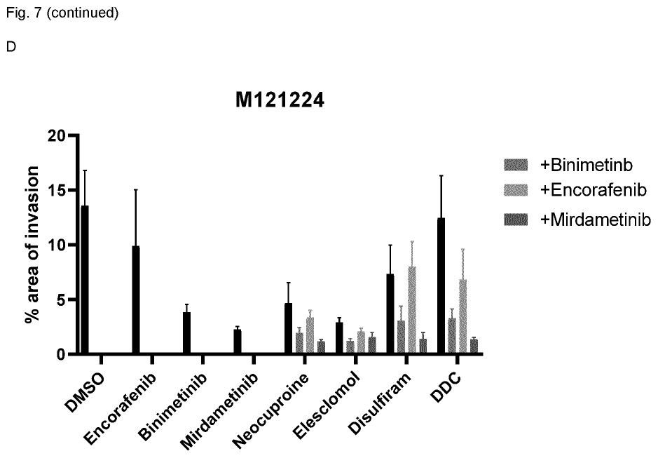

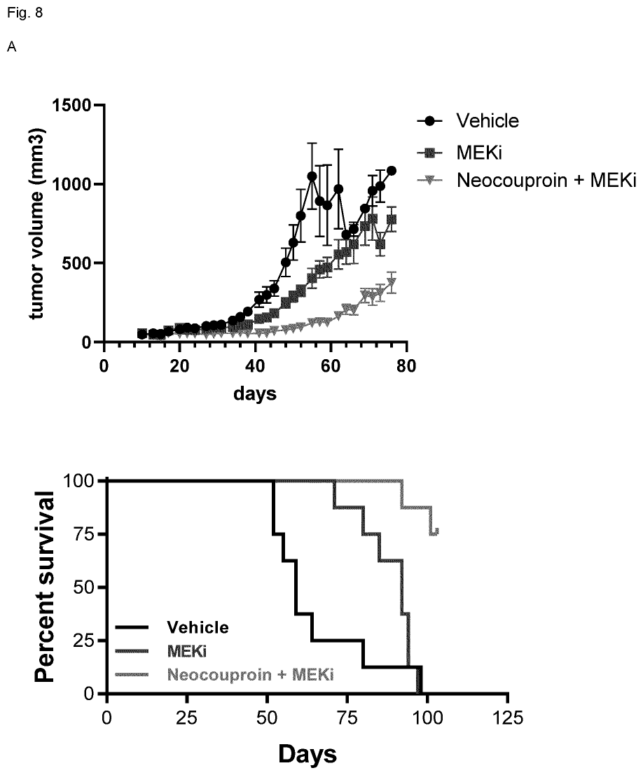

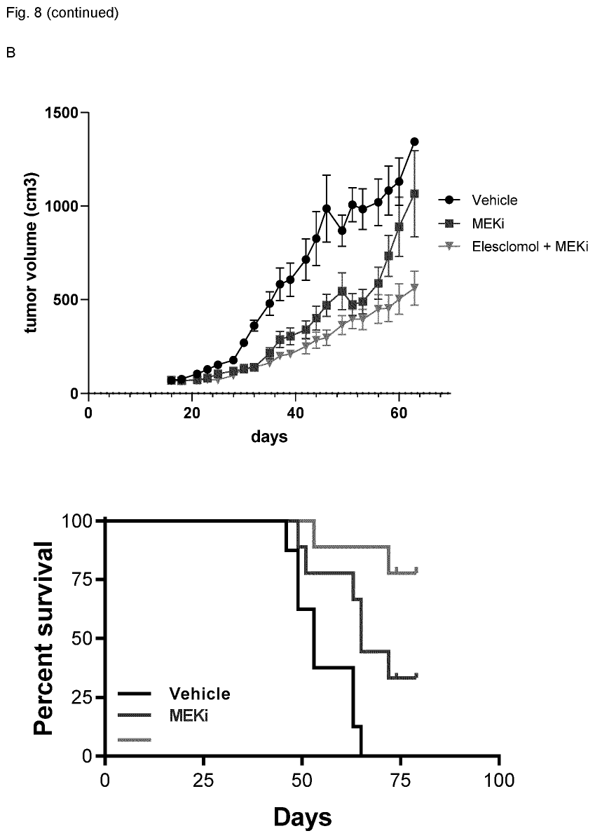

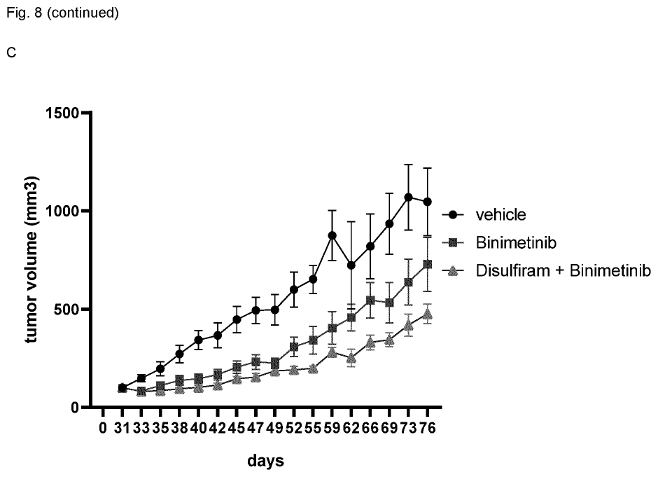

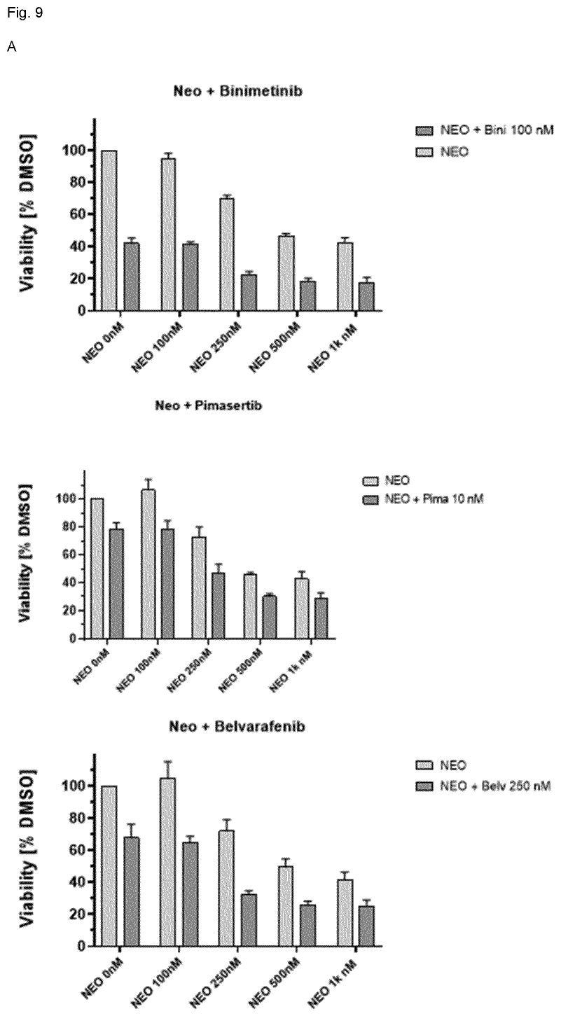

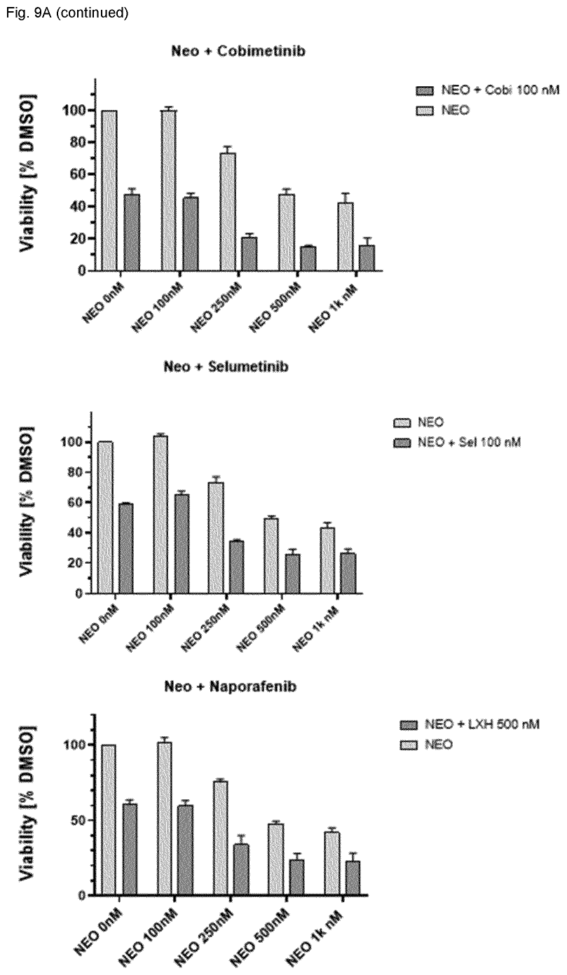

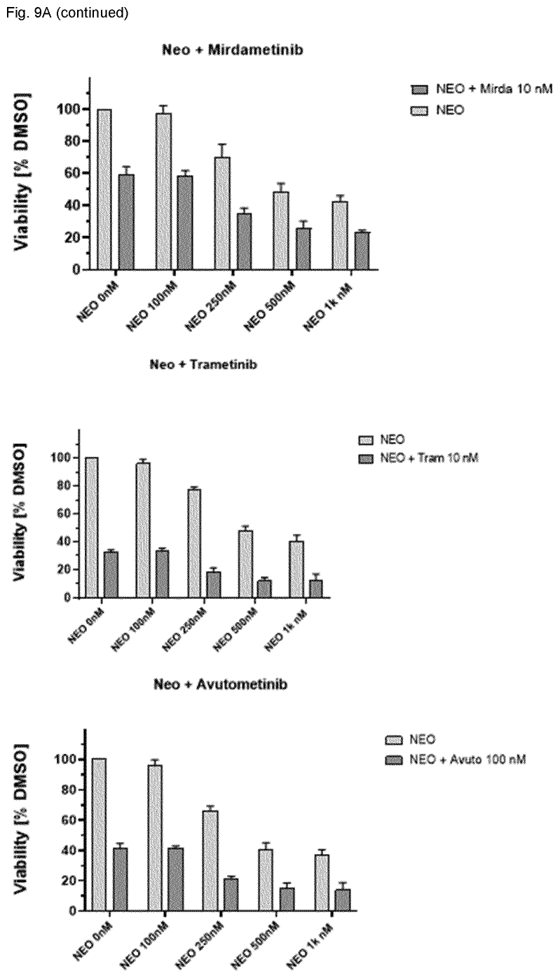

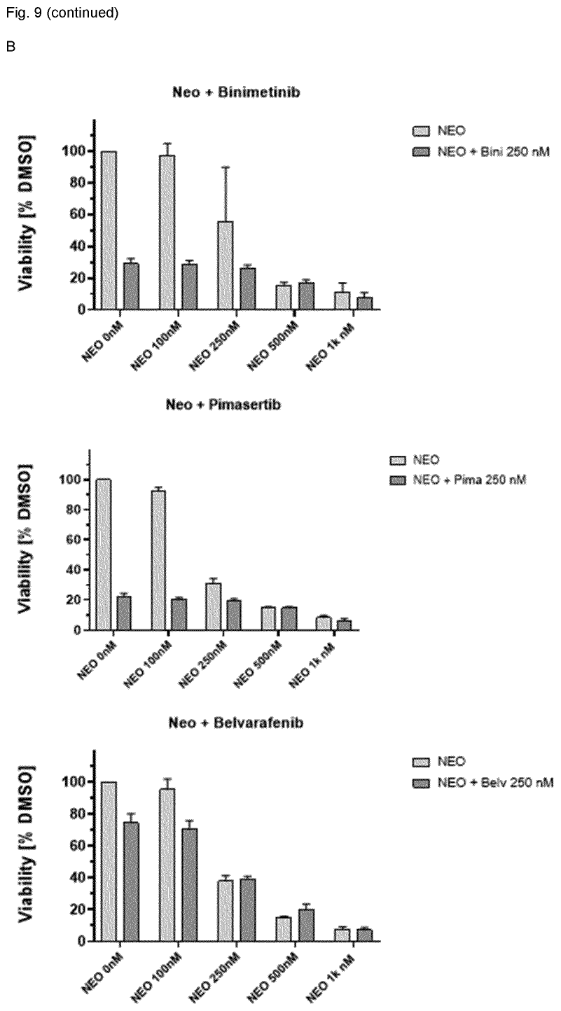

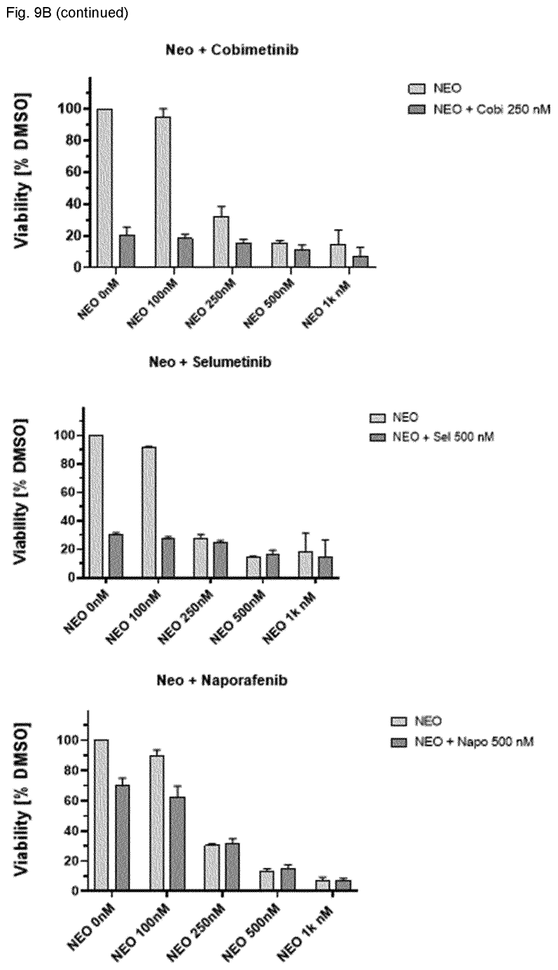

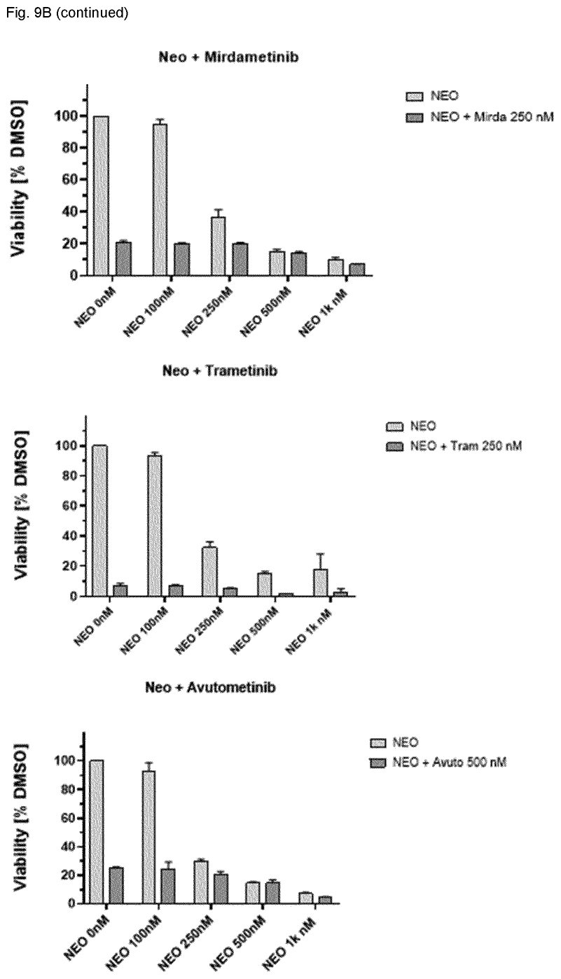

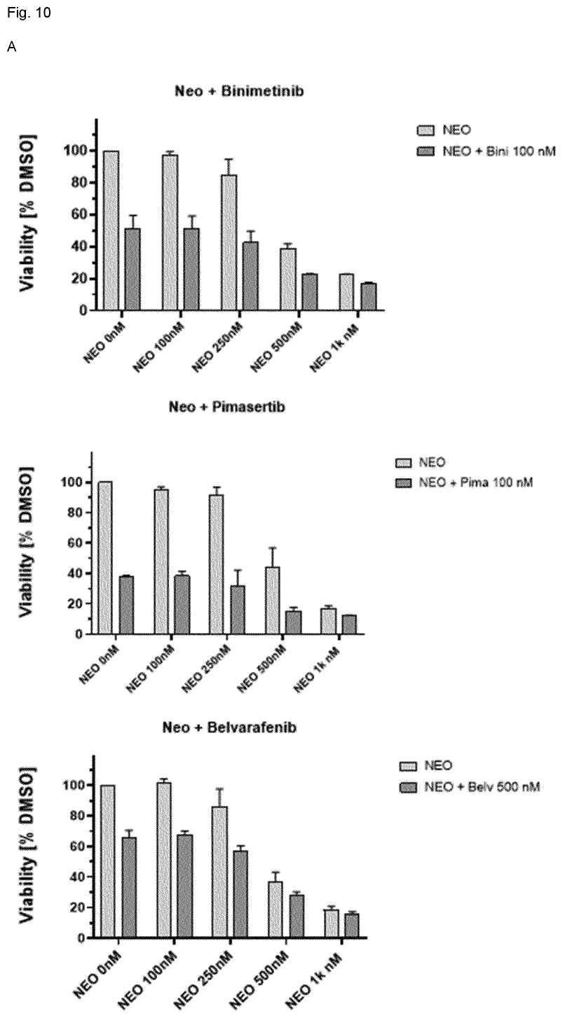

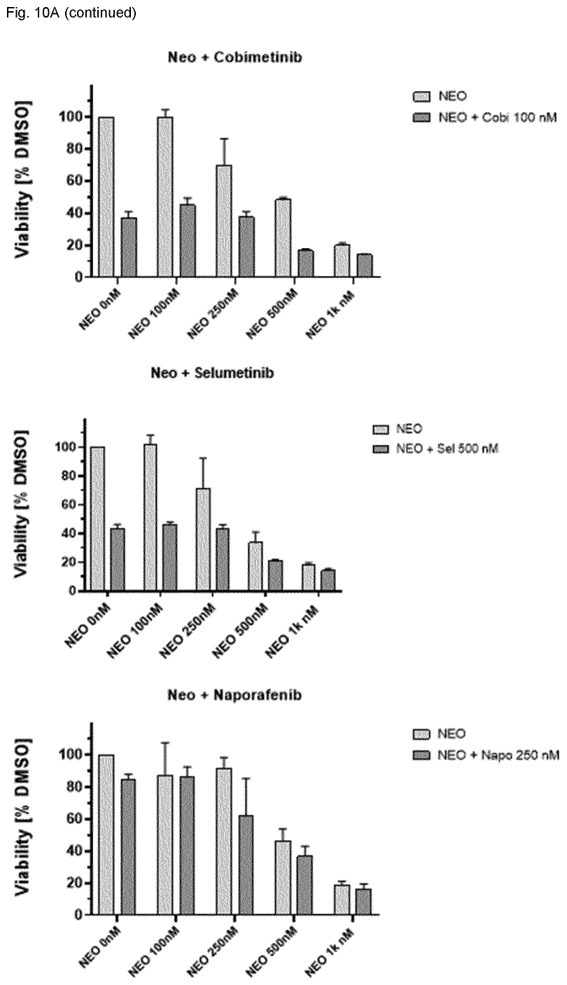

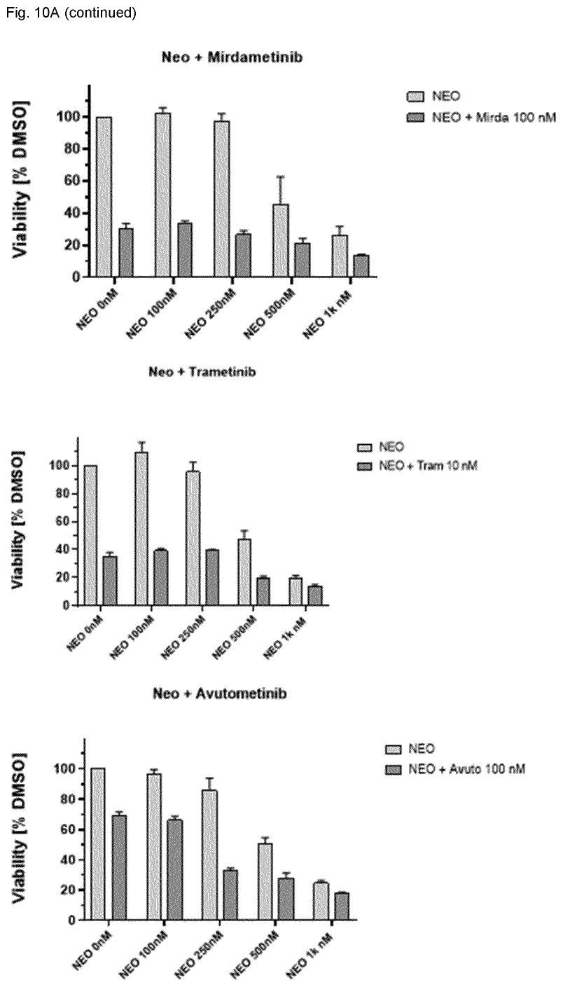

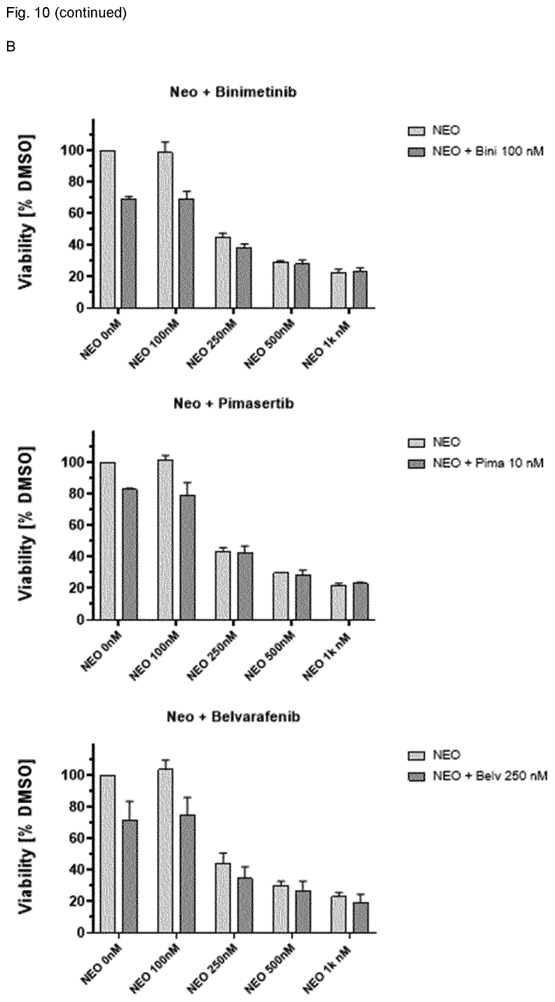

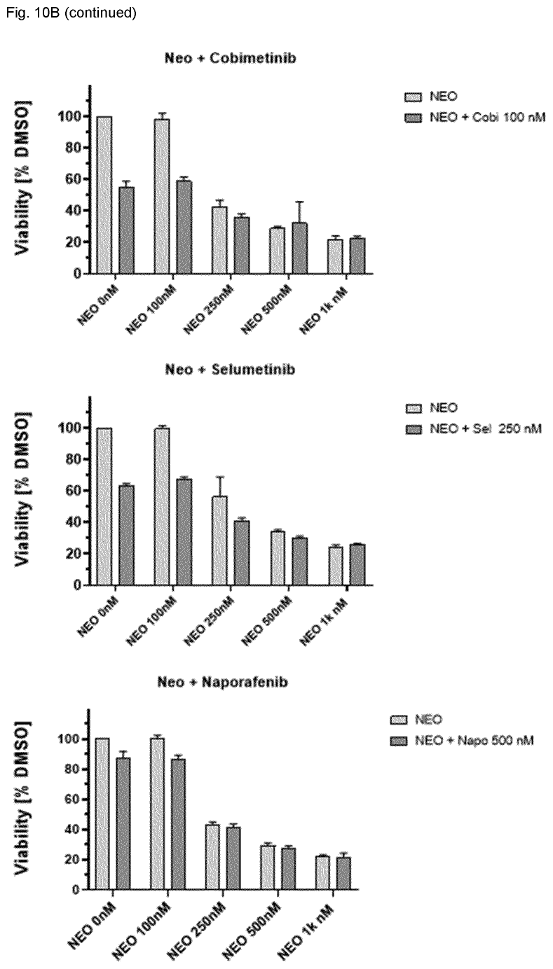

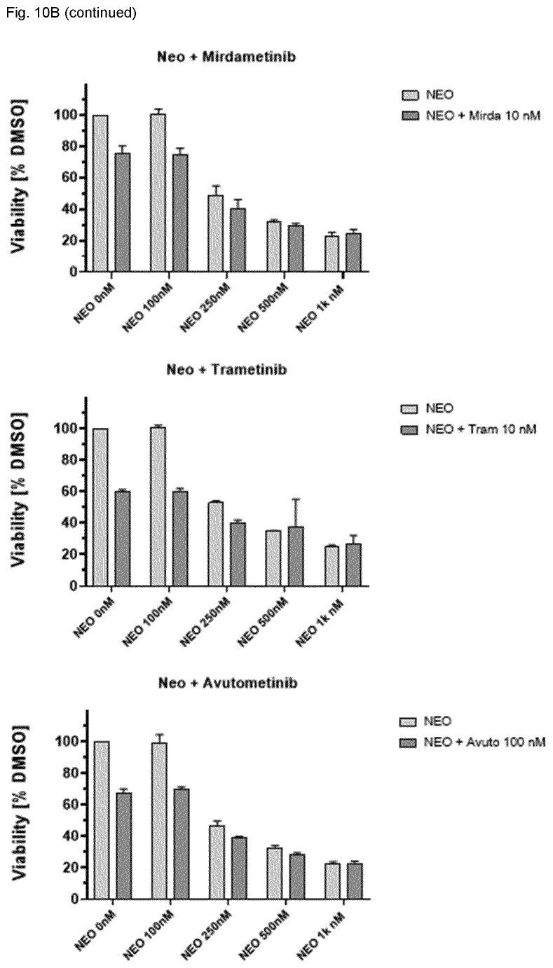

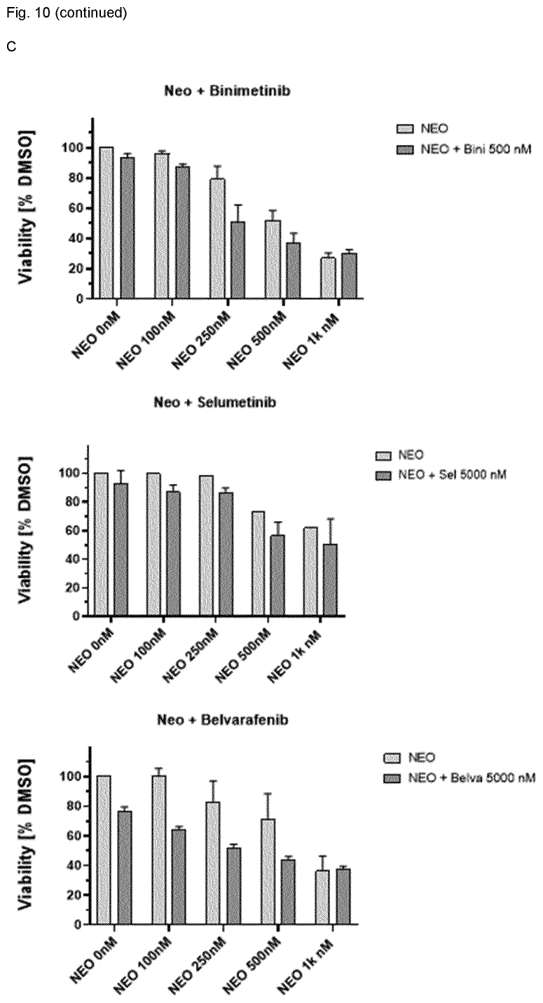

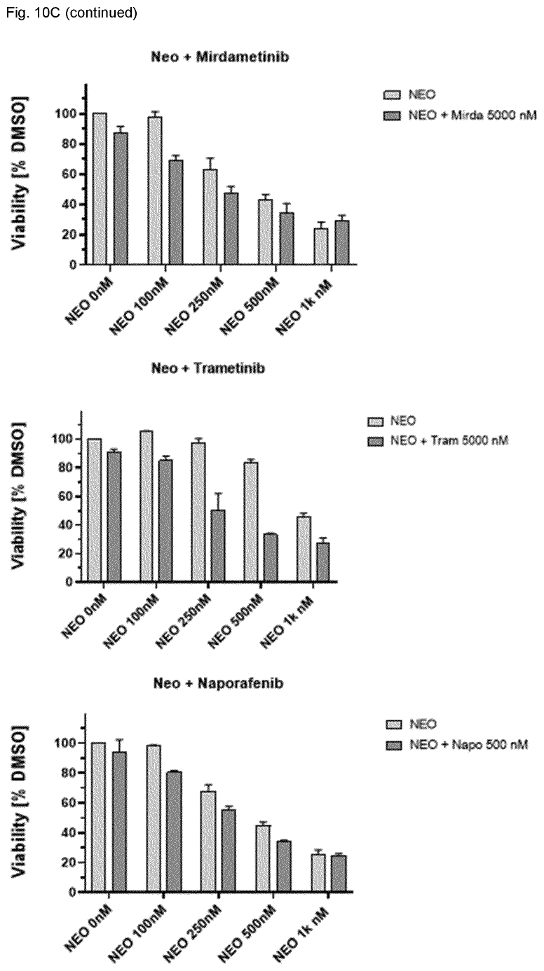

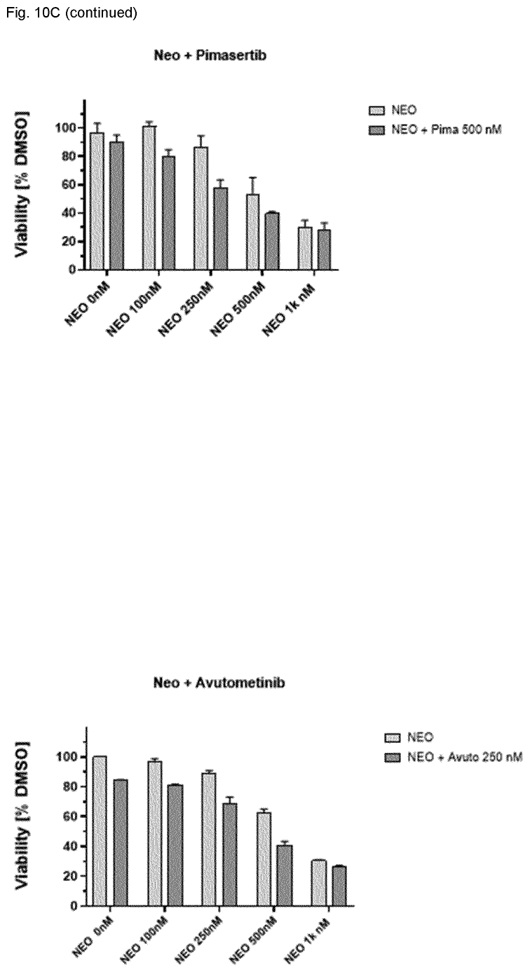

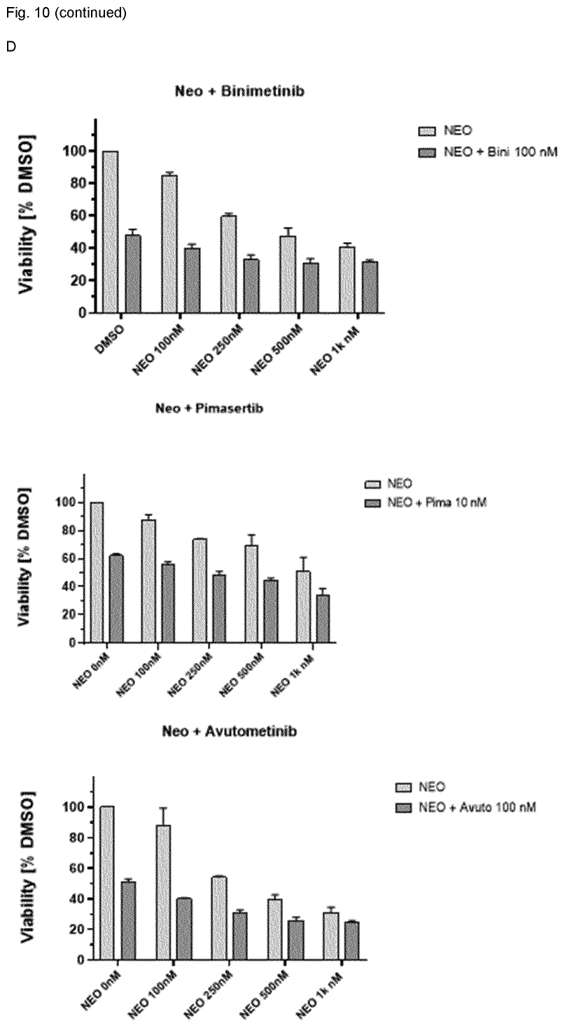

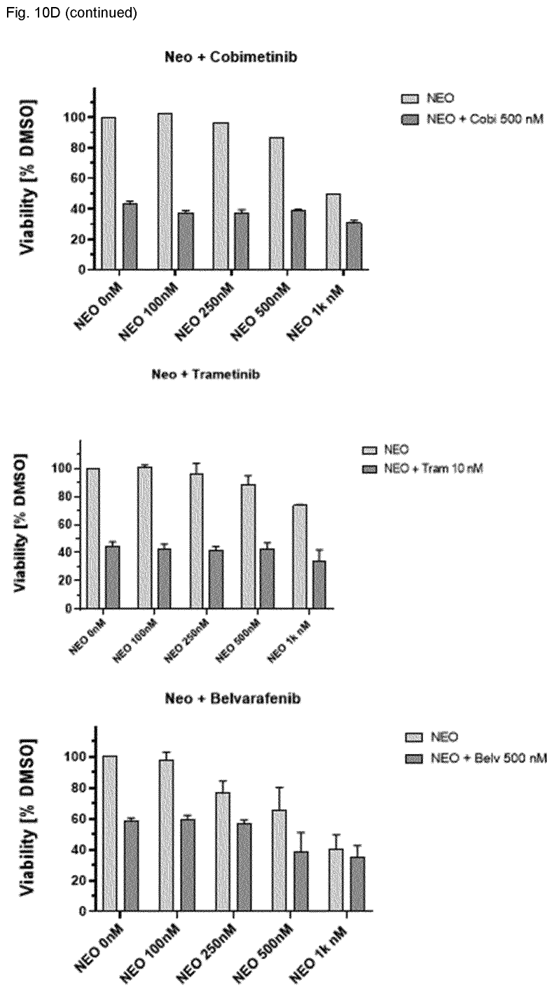

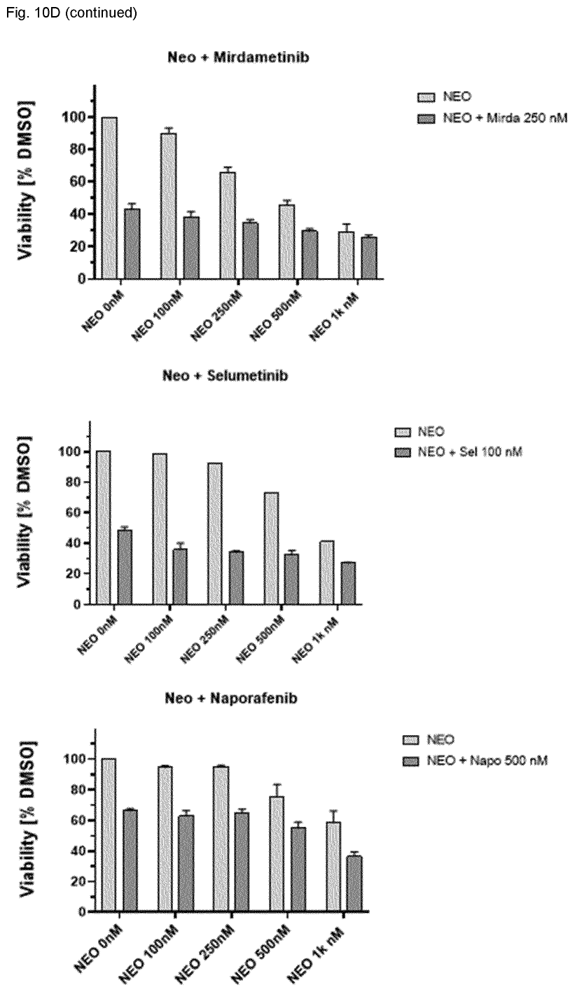

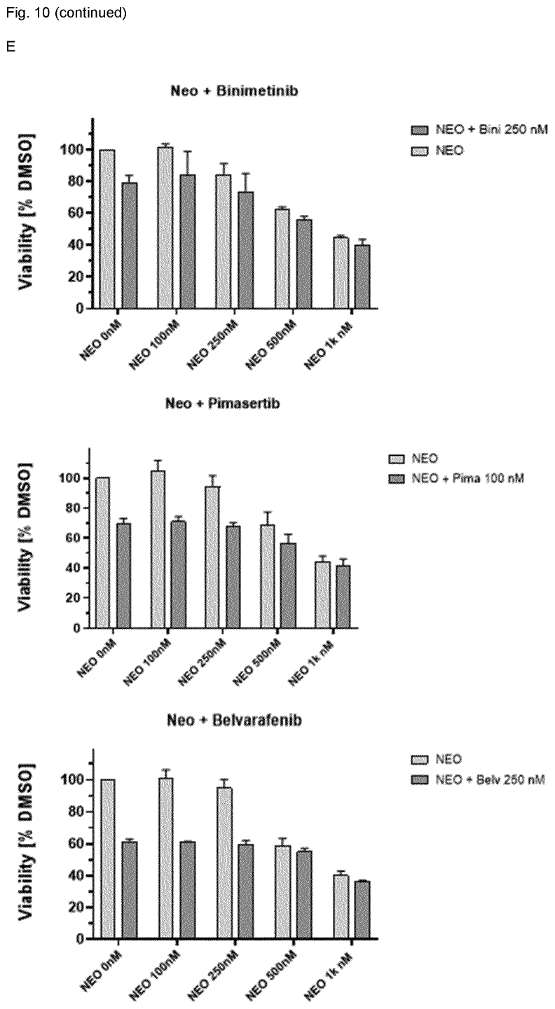

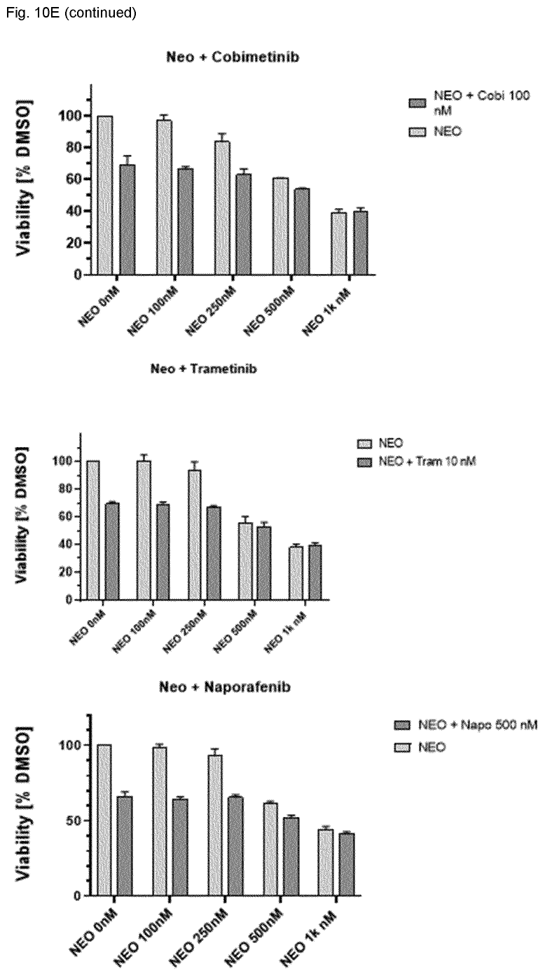

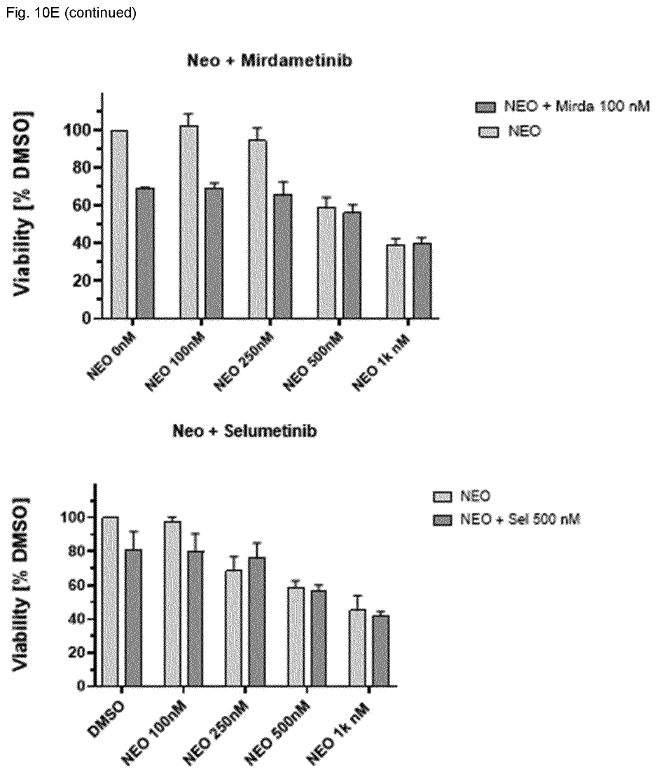

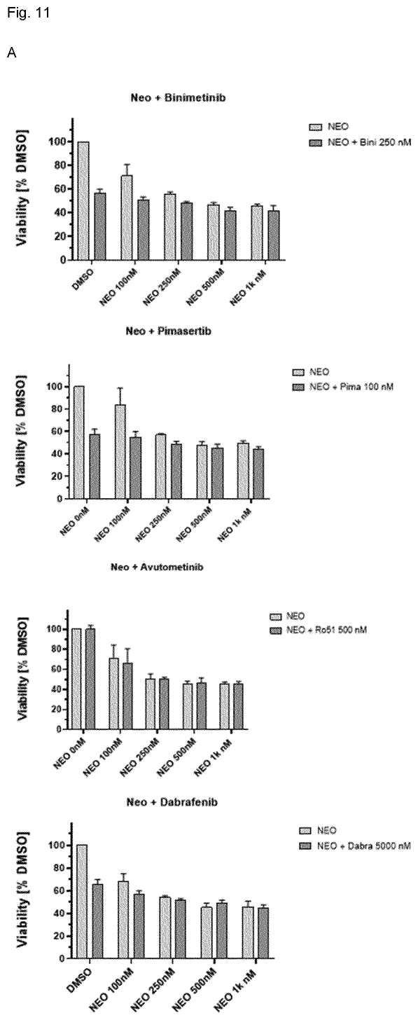

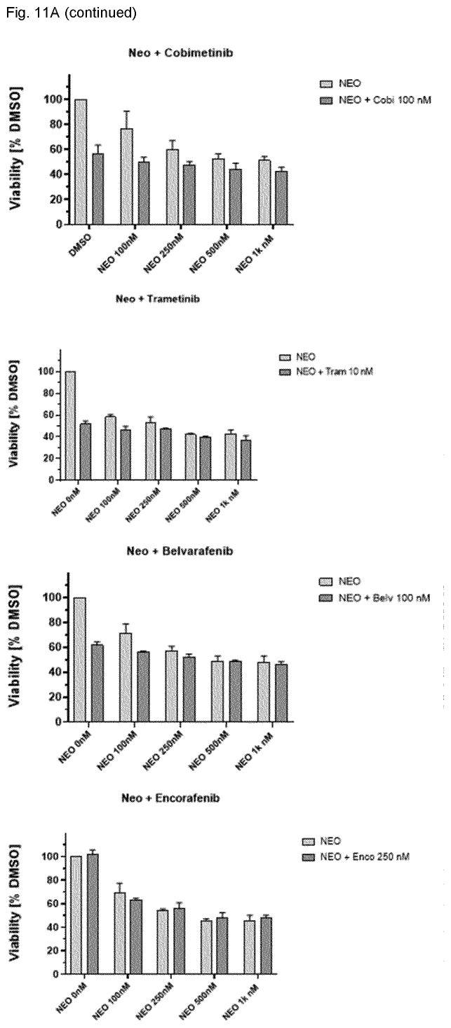

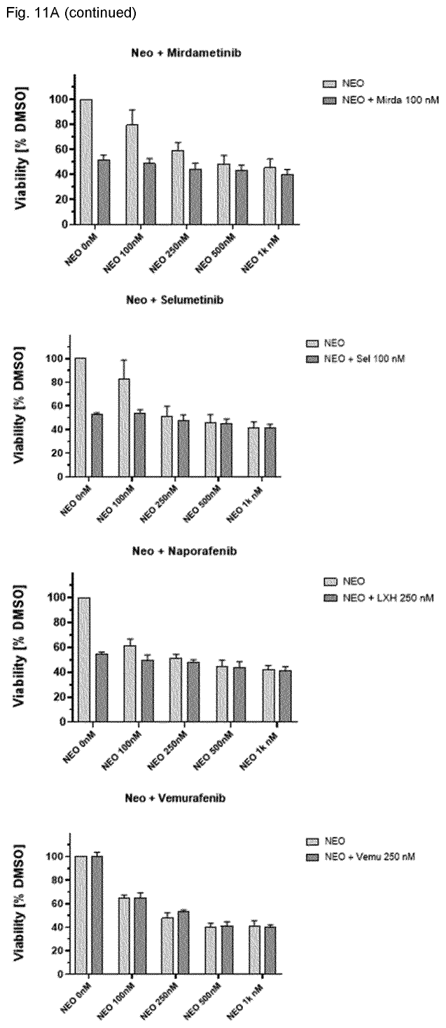

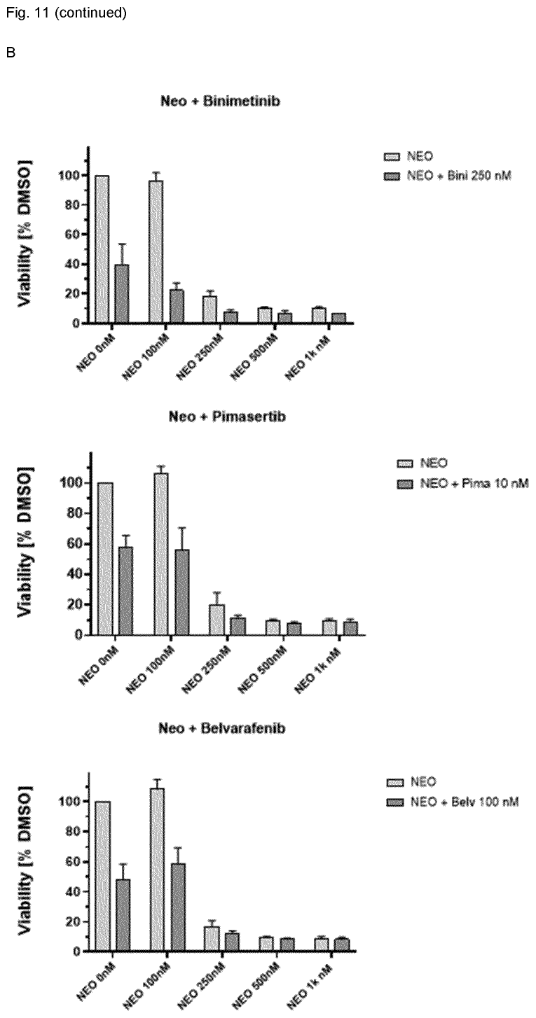

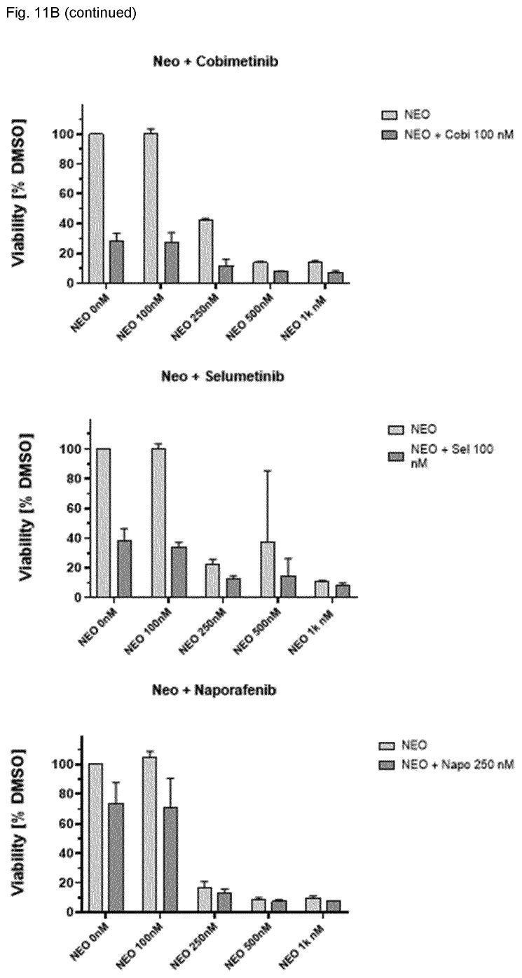

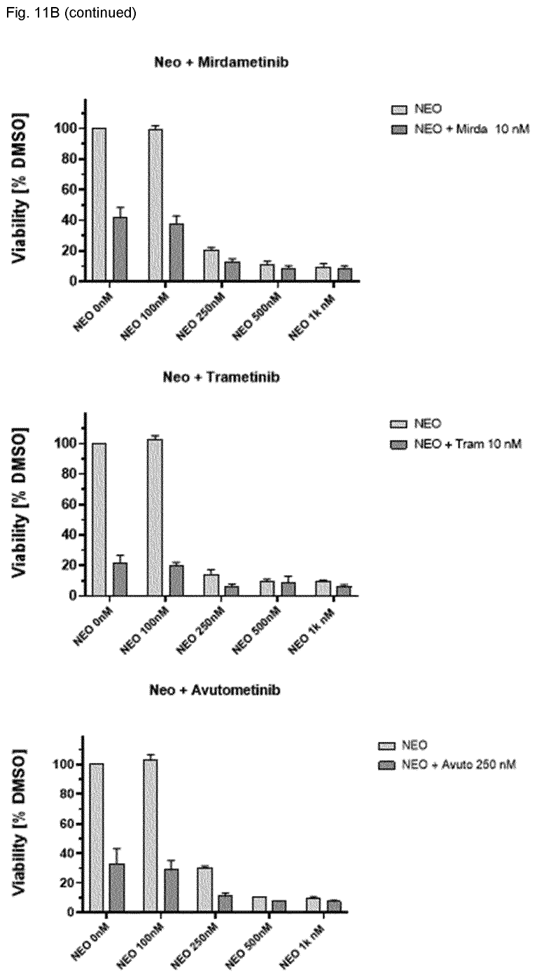

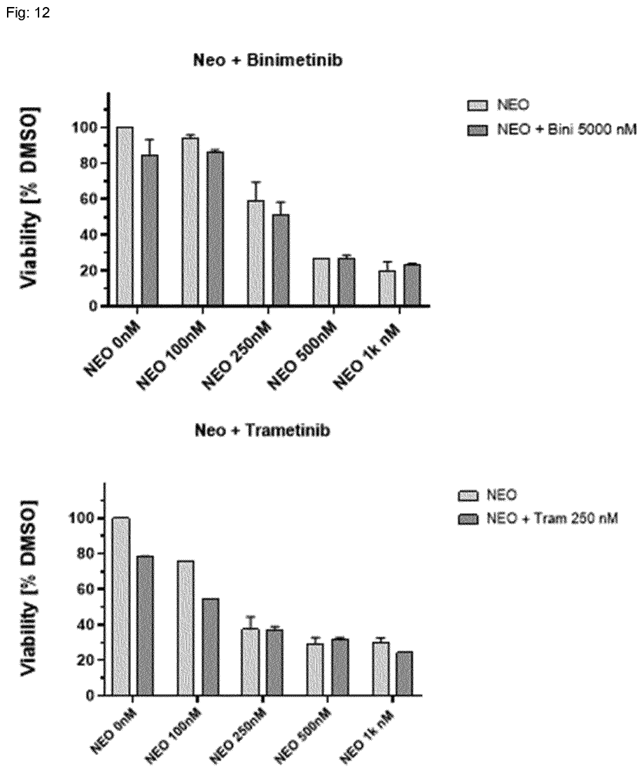

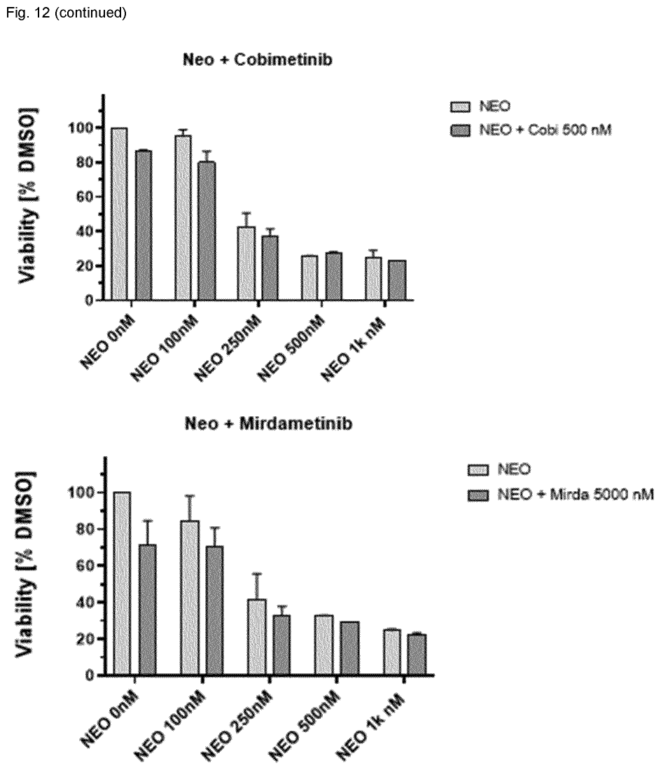

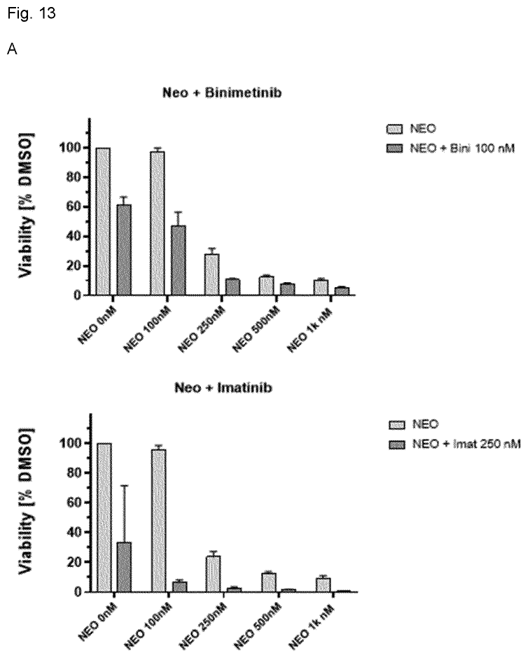

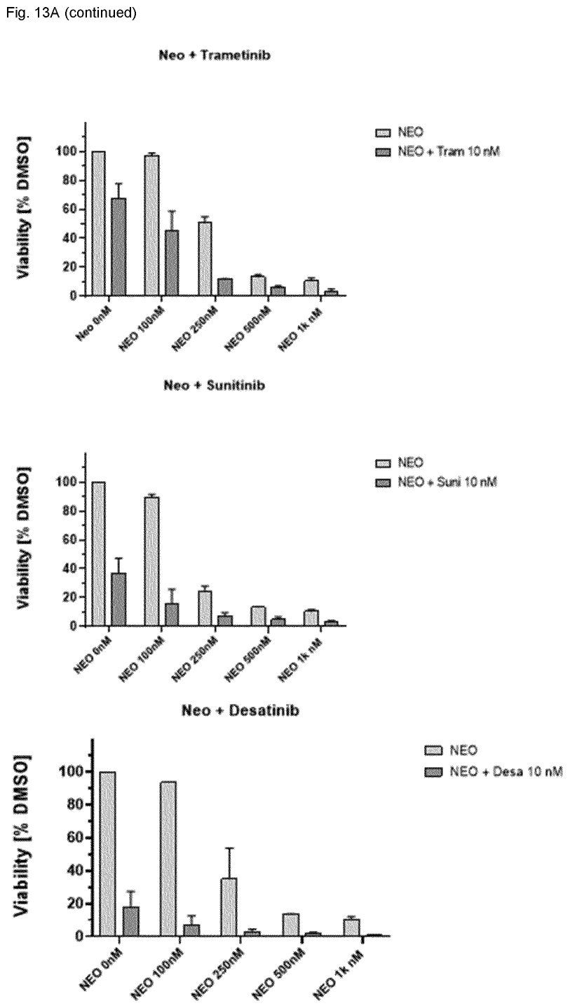

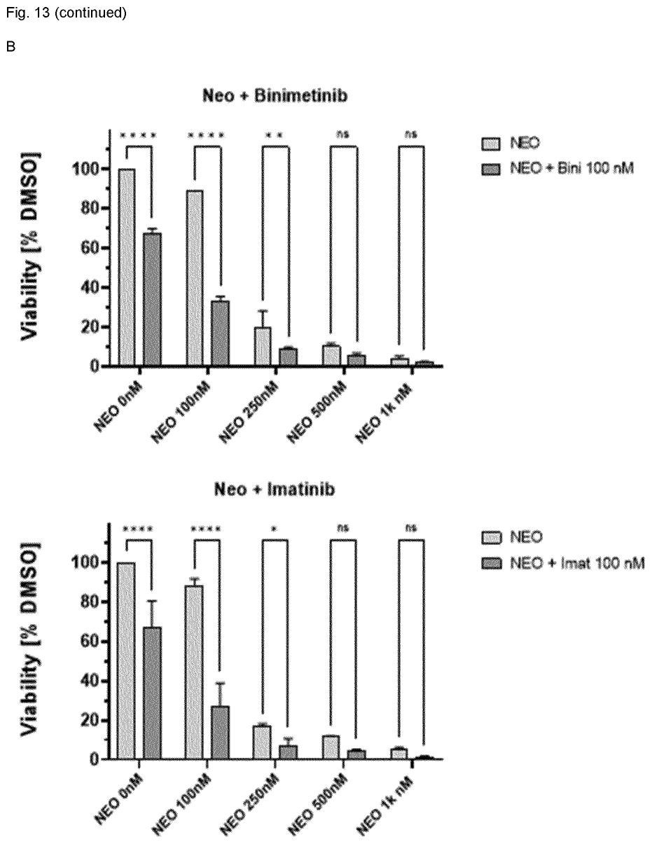

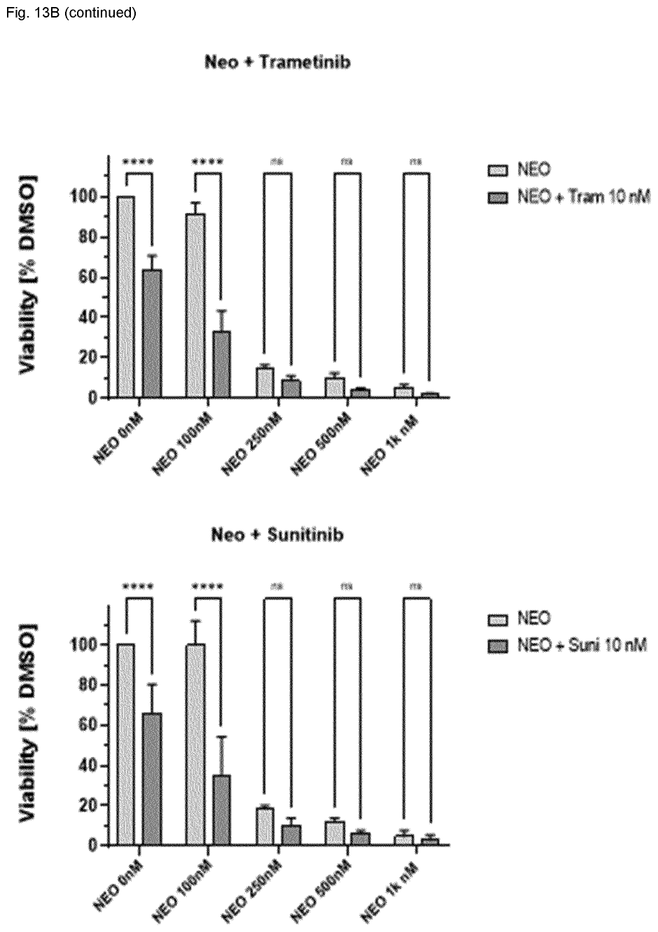

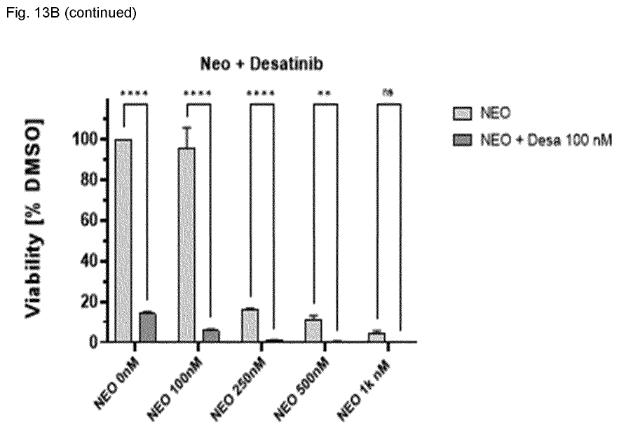

Therefore, we used Trientine to sequester Copper out of melanoma cell culture and diminished the effects of ionophoric copper-chelators used in the invention in order to prove that their mode of action on melanoma cells is copper dependent (see FIG. 4).

### Example 4: Mode of Action on Melanoma Cells is Copper-Dependent

Trientine was mixed with cell culture medium to a final concentration of 1 mM and incubated at room temperature for 1 hour. Melanoma cells were seeded in 96-well plates (cell line M130227) an let to adhere overnight. In some wells, medium was replaced with trientine-containing medium (copper-depleted). Melanoma cells were treated with neocouproine (500 nM), elesclomol (100 nM), Disulfiram (500 nM) or DDC (500 nM). Melanoma cells were incubated in the cell culture incubator at 37° C./5% CO2 for additional 72 hours. Cell viability assay was performed as described in Example 1. The % of growth inhibition (compared to DMSO treated control wells) was plotted with GraphPad Prims software.

### Example 5: Inhibition of Viability Induced in Melanoma Cell Lines by Neocuproine, Elesclomol, Disulfiram or DDC can be Rescued by the Antioxidant N-Acetyl Cysteine (NAC)

N-acetyl cysteine is an antioxidant, which is scavenging oxidative effects produced by ROS and therefore it is commonly used for the identification of ROS inducers. The inventors treated melanoma cell lines with 250 and 500 nM Neocuproine, Elesclomol, Disulfiram or DDC in the presence or absence of NAC in a proliferation assay (see Example 1) on a representative subset of melanoma cell cultures. NAC was purchased from Sigma-Aldrich (CatNr A7250) and added to the cell culture medium together with the compounds in a final concentration of 1 mM to rescue the growth inhibitory effects of Neocuproine (A), Disulfiram (C) and DDC (D) and 5 mM to rescue the growth inhibitory effect of Elesclomol (B). Melanoma cells were incubated for 72 hours 37° C./5% CO₂ and % viability was calculated in relation to vehicle treated cells. Bar graphs represent the average of repeated experiments and data are plotted with GraphPad Prism.

### Example 6: Neocuproine, Elesclomol, Disulfiram and DDC is Inducing Apoptosis in Treatment-Resistant Melanoma

It is known that while low levels of ROS can promote cell survival, high levels of ROS can activate cell death pathways like apoptosis (Redza-Dutordoire et al, 2016, Biochim Bipphys Acta). Induction of apoptosis in a cancer-cell specific manner can lead to promising therapeutic intervention, while cellular escape into a cell cycle arrest it known to be one mechanism of relapse. A distinctive feature of the early stages of apoptosis is the activation of caspase enzymes, which participate in the cleavage of protein substrates and in the subsequent disassembly of the cell. We therefore analyzed if Neocuproine, Elesclomol, Disulfiram and DDC can induce apoptosis in melanoma cells by activating Caspase activity. We used the Caspase-3/7 Detection Reagent (CellEvent™ Caspase-3/7 Green Detection Reagent, Thermo Fischer, C10423), which is a cell-permeant reagent that consists of a four-amino acid peptide (DEVD) conjugated to a nucleic acid-binding dye. During apoptosis, caspase-3 and caspase-7 proteins are activated and able to cleave the caspase 3/7 recognition sequence encoded in the DEVD peptide. Cleavage of the recognition sequence and binding of DNA by the reagent labels the apoptotic cells with a bright, fluorogenic signal that has a absorption/emission maxima of 511/533 nm. In detail, we seeded melanoma cultures (M130219, M160915, M130227 and M121224) in 6-well plates into cell culture medium (see Example 1, 6-well plates (FALCON, cat nr. 353224) and let the cells adhere over night in the cell culture incubator at 37° C./5% CO2.

Experimental table 7

| Cell culture name | Seeding density |
|---|---|
| M130219 | 1 × 10⁵ cells/well |
| M130227 | 2 × 10⁵ cells/well |
| M160915 | 3 × 10⁵ cells/well |
| M121224 | 1 × 10⁵ cells/well |

After the incubation of 48 hours with vehicle (0.1% DMSO), 1 M Neocuproine, 100 nM Elesclomol, 1 M Disulfiram or 1 μM DDC or the combination with NAC (5 mM), medium was replaced with cell culture medium containing CellEvent™ Caspase-3/7 Green Detection Reagent in a concentration according to the manufacturer's protocol. The 6-well plates were again incubated in the cell culture incubator at 37° C./5% CO₂ for 30 minutes, cells were detached from the plates by trypsinization and diluted into 5 ml of melanoma culture medium. We transferred the cell suspension into 15 ml tubes (Falcon, cat.Nr. 352096) and collected cells by centrifugation using a Heraeus Multifuge 3 S-R centrifuge centrifuge at 1500 rpm for 5 minutes. The supernatant was discarded and the cell pellet was dissolved in 200 μl of FACS buffer (PBS+0.2% fetal bovin servum, heat-inactivated (biowest cat.nr. 352096) and transferred to FACS tubes (FALCON cat.Nr. 352235). Cells were analysed by FACS using a FACS BD LSRII Fortessa. Raw data were analyzed with FlowJo software and mean fluorescent intensity per samples were plotted using GraphPad prism software were bar graphs represent the summary of technical replicates (FIG. 5 A-D). All ionophoric copper-chelators did induce apoptosis in melanoma cells and the effect could be rescued by the addition of the antioxidant NAC.

### Example 7: Efficacy in 3D Spheroids

Two-dimensional (2D) cell cultures have been demonstrated to be a valuable method for cell-based studies like drug screenings. Although this method is scientifically recognized, in recent years three-dimensional (3D) cell culture and tissue models have been evolved especially in the field of drug discovery. Under physiological conditions, cells in their normal in vivo environment are always surrounded by other cells and/or compounds of extracellular matrix (ECM) forming a 3D structure. Therefore, 3D cell culture models mimic features of an in vivo situation like cell-cell interactions and drug diffusion barriers. Therefore, the inventors investigated the effects of Neocuproine, Elesclomol, Disulfiram and DDC in combination with the MEK inhibitors Binimetinib (MEK162, Selleckchem.com) and Mirdametinib (supplied by SpringWorks Therapeutics, Stamford, Connecticut, USA) on melanoma cells by 3D spheroid invasion assay.

Briefly, a 96-well flat bottom plate was coated with 1.5% Noble Agar (BD, cat Nr. 214 220) diluted in PBS. The agar polymerized for 1 h under UV-light in the laminar-flow hood followed by 2h incubation in the cell culture incubator at 37° C./5% CO2. 10.000 cell/well of M121224 or M160915 were seeded in 100 μl of cell culture medium on top of the agar which hinders the cells to attach to the bottom of the well. After additional incubation for 24h-72h at 37° C./5% CO2, cells formed round-shaped spheres. The spheres were transferred in 1.5 ml Eppendorf tubes, let precipitate and residual medium was removed. Collagen Type I was mixed with medium, fetal bovine serum, L-glutamine, sodium pyruvate and bicarbonate (see table below) and 100 I were mixed into the Eppendorf tube containing the spheroid and transferred to a 96-well plate again pre-coated with 1.5% Noble Agar as described above. The plate was then incubated for 2 h at 37° C./5% CO₂ to polymerize the collagen matrix. Embedded spheroids were overlaid with cell culture medium (100 I).

Experimental table 8:

| Amount [μL] | Substance |
|---|---|
| 7.5 | Minimum Essential Medium Eagle (EMEM) (10xEMEM w/ EBSS); |
| | Gibco by Life Technologies (USA) |
| 1.5 | L-Glutamine (200 nM); Gibco by Life Technologies (USA) |
| 7.5 | Fetal bovine serum (FBS); Gibco by Life Technologies (USA) |
| 27.5 | Collagen Type I (Rat Tail); Corning (USA) (Cat nr. 11563550) |
| 27.5 | Phosphate Buffered Saline System (PBS) Dulbecco (w/o Ca²⁺ w/o Mg²⁺); |
| | Gibco by Life Technologies (USA) |
| 3.75 | NaHCO₃ (7.5%); Sigma-Aldrich (USA) |

After 24h-72h when first signs of invasion from the sphere was observed the investors started the treatment by replenishing the melanoma culture medium overlaying the spheroids with melanoma culture medium with 0.5, 1 or 5 M of Neocuproine or 500 nM MEK162 (Selleckchem, CatNr S7007) or LGX818 (Selleckchem, CatNr S7108) or the combination. Treatment was repeated after 72h for additional 48h. Spheroids were stained with two different fluorescent dyes: Calcein-Green-AM (SigmaAldrich CatNr 17783) and ethidium homodimers (SigmaAldrich CatNr 46043-1 MG-F) which stain live and dead cells, respectively. Calcein-Green-AM is only converted into its fluorescent form by living cells upon intracellular esterase activity, whereas ethidium homodimers are taken up passively by cells with leaking membranes (dead cells) and binds to DNA. After adding the dyes, the plate was incubated at 37° C./5% CO₂ for 1 h and the spheres were imaged with a Leica TCS LSI microscope (Calcein-Green Ex494 nm, Ethidium homodimers Ex 528 nm). FIG. 6 A shows representative images of 3D melanoma spheroids treated with ionophoric copper chelators or MEK inhibitors (Binimetinib or Mirdametinib) or the combination. % area of invasion is plotted as an average of 4 spheres analysed by ImageJ software using “analyse particles” measurements limited to set threshold. We that combination of an ionophoric copper chelator with a MEK inhibitor did significantly decrease the invasion of melanoma cells into the collagen matrix (FIG. 4B). We found no significant differences in the effect of the MEK inhibitors.

### Example 8: Efficacy In Vivo

Here we used cell culture M130227, a MEK inhibitor resistant cell culture, sensitive for ionophoric copper chelators Neocuproine, Elesclomol, Disulfiram and DDC. Animal experiments were accepted by the veterinary office of the Kanton Zurich, Switzerland under the license nr. ZH095/17.
1. We did inject M130227 melanoma cells (500.000 per mouse) mixed into 100 μl PBS/Matrigel (Corning Cat.Nr.356234) in a ratio of 1:1 into the flank of immunocompromised mice (strain Balb/c) and observed the initiation of tumour growth after about 2 weeks. We started the treatment when tumors were palpable. We randomized mice into 4 groups of 8 individuals and treated them as following:
2. Vehicle (200 I 1.5% DMSO in PBS) injected 3 times per week i.p. (intra peritoneal) and 200 μl of PBS with 0.5% Tweeb-80 and 1% carboxymethylcellulose by oral gavage every working day
3. Binimetinib (30 mg/kg, dissolved in 200 μl of PBS with 0.5% Tweeb-80 and 1% carboxymethylcellulose) by oral gavage every working day.
4. The combination of 2. And Neocuproine (2 mg/kg, dissolved in 200 μl 1.5% DMSO in PBS) injected 3 times per week i.p.

Tumors were measured every second day and tumour volume was calculated using the following formula: Tumor Volume (mm³)=(length(mm)×width(mm)²)/2. We plotted the tumor volumes using GraphPad prism software.

The data are showing the partial response to the MEK inhibitor Binimetinib which was also reported in clinical studies. The combination of Neocuproine and Binimetinib was significant superior to all other treatment groups. Kaplan-Meier survival curves were plotted using GraphPad Prism software. Kaplan-Meier curves are used in medical research frequently to measure the fraction of individuals living for a certain amount of time after treatment. Here we demonstrate the survival of individual mice per group after treatment initiation.

According to the above-described experimental set-up we conducted further mice experiments with EPO Berlin-Buch GmbH, Germany, under contract research. Here we used either Elesclomol or Disulfiram in combination with Binimetinib and animals were treated as following:
1. Vehicle (200 I 1.5% DMSO in PBS) injected 3 times per week i.p. (intra peritoneal) and 200 I of PBS with 0.5% Tweeb-80 and 1% carboxymethylcellulose by oral gavage every working day
2. MEK162 Binimetinib (30 mg/kg, dissolved in 200 I of PBS with 0.5% Tweeb-80 and 1% carboxymethylcellulose) by oral gavage every working day
3. The combination of 2. And Neocuproine (2 mg/kg, dissolved in 200 l 1.5% DMSO in PBS) injected 3 times per week i.p.

### Example 9: Combinations of Copper Ionophores with MAPK-Pathway Inhibitors in NRAS Mutated Cancers have a Beneficial Efficacy

The efficacy of viability reduction of neocuproine alone, compared to MAPK-pathway inhibitors (available clinically or undergoing clinical trials) alone, and the combination of neocuproine and the MAPK-pathway inhibitors, were assessed in NRAS mutated cancer cells of different cancer origin using the Resazurin viability assay:

The cancer cells were seeded into 96-well plates (seeding densities see experimental table 2) in 80 μl of their appropriate cell culture medium (according to experimental table 2). Cells were then let to adhere overnight. Neocuproine (STO13881, TimTec, Cat Nr. MFCD00004973; CAS: 484-11-7) and the MAPK-pathway inhibitors Binimetinib (MEK inhibitor, Cat. No. S7007, Selleckchem), Cobimetinib (MEK inhibitor, Cat. No. S8041, Selleckchem), Mirdametinib (MEK inhibitor, Cat. No. S1036, Selleckchem), Pimasertib (MEK inhibitor, Cat. No. S1475, Selleckchem), Trametinib (MEK inhibitor, Cat. No. S2673, Selleckchem), Selumetinib (MEK inhibitor, Cat. No. S1008, Selleckchem), Belvarafenib (panRAF inhibitor, Cat. No. S8853, Selleckchem), Naporafenib (panRAF inhibitor, Cat. No. S8745, Selleckchem), Avutometinib (RAF/MEK inhibitor, Cat. No. S7170, Selleckchem) were diluted in DMSO (dimethylsufoxide, Cat. No. 67-68-5) to a 1000-fold higher stock solution than the final concentration in the culture wells, starting with 10 mM, using a serial dilution schedule. The drugs were then further diluted 1:100 in cell culture medium in a separate 96-well plate. From this 1:100 dilution plate, 10 μl were transferred to the 96-well plate containing the cancer cells, resulting in the desired 1:1000 final concentrations from the 1000-fold higher stock solutions. Neocuproine only wells were filled with 10 μl of cell culture medium, resulting in a total of 100 μl cell culture medium per well, equal to the total of 100 μl cell culture medium in the case of neocuproine+MAPK-pathway inhibitor combination wells. Neocuproine (NEO) final concentrations were 0 nM (DMSO vehicle control), 100 nM, 250 nM, 500 nM and 1000 nM (1 k nM). Each of the MAPK-pathway inhibitors was tested at various concentrations, with the most representative for each cell line shown in this application. The final concentrations were 1 nM, 10 nM, 100 nM, 250 nM, 500 nM, 1000 nM, 5000 nM and 10000 nM. The solvent (here DMSO) served as control and was added in the appropriate concentration to the control wells. After 72h cell viability was measured with a standard viability assay using resazurin (7-hydroxy-3H-phenoxazin-3-one 10-oxide, CAS No. 550-82-3, 0.15 mg/ml in PBS). Per 96-well culture plate 1 ml of resazurin solution was mixed with 9 ml melanoma cell culture medium. The medium of the melanoma cell containing 96-well plate was exchanged with 100 μl/well resazurin/medium mixture. Plates were then incubated in the tissue culture incubator for 2-4 h at 37° C./5% CO2 and fluorescent intensity was measured in a standard fluorescence plate reader (Tecan) with the following parameter: Excitation 535 nm/Emission 595 nm. Data are analysed by GraphPad Prism Software. DMSO treated wells are set to 100% viability and IC50 values are calculated accordingly.

FIG. 9 shows the beneficial effect of the addition of neocuproine (NEO) to MAPK-pathway inhibitors that are currently available clinically or undergoing clinical trials. Compared to the DMSO vehicle control (NEO 0 nM), the MAPK-pathway inhibitor only (NEO 0 nM+MAPK-pathway inhibitor at indicated concentration) is capable of reducing the viability of the cancer cell line. However, if neocuproine is combined with the MAPK-pathway inhibitor, higher reduction of cancer cell viability is achieved, compared to the MAPK-pathway inhibitor only treatment. The neocuproine+MAPK-pathway inhibitor combination is therefore beneficial over the MAPK-pathway inhibitor monotreatment.

### Example 10: Combinations of Copper Ionophores with MAPK-Pathway Inhibitors in KRAS Mutated Cancers have a Beneficial Efficacy

The efficacy of viability reduction of neocuproine alone, compared to MAPK-pathway inhibitors (available clinically or undergoing clinical trials) alone, and the combination of neocuproine and the MAPK-pathway inhibitors, was assessed in KRAS mutated cancer cells of different cancer origin using the Resazurin viability assay. For assay procedure, see assay procedure in example 9.

FIG. 10 shows the beneficial effect of the addition of neocuproine (NEO) to MAPK-pathway inhibitors that are currently available clinically or undergoing clinical trials. Compared to the DMSO vehicle control (NEO 0 nM), the MAPK-pathway inhibitor only (NEO 0 nM+MAPK-pathway inhibitor at indicated concentration) is capable of reducing the viability of the cancer cell line. However, if neocuproine is combined with the MAPK-pathway inhibitor, higher reduction of cancer cell viability is achieved, compared to the MAPK-pathway inhibitor only treatment. The neocuproine+MAPK-pathway inhibitor combination is therefore beneficial over the MAPK-pathway inhibitor monotreatment.

### Example 11: Combinations of Copper Ionophores with MAPK-Pathway Inhibitors in BRAF Mutated Cancers have a Beneficial Efficacy

The efficacy of viability reduction of neocuproine alone, compared to MAPK-pathway inhibitors (available clinically or undergoing clinical trials) alone, and the combination of neocuproine and the MAPK-pathway inhibitors, was assessed in BRAF mutated cancer cells of different cancer origin using the Resazurin viability assay. For assay procedure, see assay procedure in example 9. As BRAF-inhibitors are clinically applied in BRAF mutated cancers, in certain examples Dabrafenib (BRAF inhibitor, Cat. No. S2807, Selleckchem), Encorafenib (BRAF inhibitor, Cat. No. S7108, Selleckchem) and Vemurafenib (BRAF inhibitor, Cat. No. S1267, Selleckchem) were used additionally.

FIG. 11 shows the beneficial effect of the addition of neocuproine (NEO) to MAPK-pathway inhibitors that are currently available clinically or undergoing clinical trials. Compared to the DMSO vehicle control (NEO 0 nM), the MAPK-pathway inhibitor only (NEO 0 nM+MAPK-pathway inhibitor at indicated concentration) is capable of reducing the viability of the cancer cell line. However, if neocuproine is combined with the MAPK-pathway inhibitor, higher reduction of cancer cell viability is achieved, compared to the MAPK-pathway inhibitor only treatment. The neocuproine+MAPK-pathway inhibitor combination is therefore beneficial over the MAPK-pathway inhibitor monotreatment.

### Example 12: Combinations of Copper Ionophores with MAPK-Pathway Inhibitors in NF1 Mutated Cancers have a Beneficial Efficacy

The efficacy of viability reduction of neocuproine alone, compared to MAPK-pathway inhibitors (available clinically or undergoing clinical trials) alone, and the combination of neocuproine and the MAPK-pathway inhibitors, was assessed in BRAF mutated cancer cells of different cancer origin using the Resazurin viability assay. For assay procedure, see assay procedure in example 9.

FIG. 12 shows the beneficial effect of the addition of neocuproine (NEO) to MAPK-pathway inhibitors that are currently available clinically or undergoing clinical trials. Compared to the DMSO vehicle control (NEO 0 nM), the MAPK-pathway inhibitor only (NEO 0 nM+MAPK-pathway inhibitor at indicated concentration) is capable of reducing the viability of the cancer cell line. However, if neocuproine is combined with the MAPK-pathway inhibitor, higher reduction of cancer cell viability is achieved, compared to the MAPK-pathway inhibitor only treatment. The neocuproine+MAPK-pathway inhibitor combination is therefore beneficial over the MAPK-pathway inhibitor monotreatment.

### Example 13: Combinations of Copper Ionophores with MAPK-Pathway Inhibitors in cKIT Mutated Cancers have a Beneficial Efficacy

The efficacy of viability reduction of neocuproine alone, compared to tyrosine kinase inhibitors (available clinically or undergoing clinical trials) alone, and the combination of neocuproine and the MAPK-pathway inhibitors, was assessed in cKIT mutated cancer cells using the Resazurin viability assay. For assay procedure, see assay procedure in example 9. For cKit mutated cells direct MAPK-pathway inhibitors (MEK inhibitors) and the tyrosine kinase inhibitors (TKi) imatinib (TKi, Cat. No. S1026, Selleckchem), Sunitinib (TKi, Cat. No. S1042, Selleckchem) and Desatinib (TKi, Cat. No. S1042, Selleckchem) were used. These are inhibitors of the receptor tyrosine kinase cKIT, a MAPK-pathway activating kinase, upstream of the MAPK-pathway.

FIG. 13 shows the beneficial effect of the addition of neocuproine (NEO) to MAPK-pathway inhibitors or tyrosine kinase inhibitors that are currently available clinically or undergoing clinical trials. Compared to the DMSO vehicle control (NEO 0 nM), the MAPK-pathway inhibitor or tyrosine kinase inhibitor only (NEO 0 nM+MAPK-pathway inhibitor or NEO 0 nM+tyrosine kinase inhibitor at indicated concentration) is capable of reducing the viability of the cancer cell line. However, if neocuproine is combined with the MAPK-pathway inhibitor or tyrosine kinase inhibitor, higher reduction of cancer cell viability is achieved, compared to the MAPK-pathway inhibitor or tyrosine kinase inhibitor only treatment. The neocuproine+MAPK-pathway inhibitor or neocuproine+tyrosine kinase inhibitor combination is therefore beneficial over the tyrosine kinase inhibitor monotreatment.

### LITERATURE

Chen, S., et al., Syntheses and antitumor activities of N′1,N′3-dialkyl-N′1,N′3-di-(alkylcarbonothioyl) malonohydrazide: the discovery of elesclomol. Bioorg Med Chem Lett, 2013. 23(18): p. 5070-6.
Berkenblit, A., et al., Phase I clinical trial of STA-4783 in combination with paclitaxel in patients with refractory solid tumors. Clin Cancer Res, 2007. 13(2 Pt 1): p. 584-90.
O'Day, S., et al., Phase II, randomized, controlled, double-blinded trial of weekly elesclomol plus paclitaxel versus paclitaxel alone for stage IV metastatic melanoma. J Clin Oncol, 2009. 27(32): p. 5452-8.
O'Day, S. J., et al., Final results of phase III SYMMETRY study: randomized, double-blind trial of elesclomol plus paclitaxel versus paclitaxel alone as treatment for chemotherapy-naive patients with advanced melanoma. J Clin Oncol, 2013. 31(9): p. 1211-8.
Babak, M. V. and D. Ahn, Modulation of Intracellular Copper Levels as the Mechanism of Action of Anticancer Copper Complexes: Clinical Relevance. Biomedicines, 2021. 9(8).

1. A method for treatment of a cancer comprising,
administering to a patient in need thereof a therapeutically effective amount of neocuproine,
wherein the cancer is characterized by cells having
a. an NRAS mutation; and/or
b. a KRAS mutation; and/or
c. an HRAS mutation; and/or
d. a BRAF mutation; and/or
e. a cKIT mutation; and/or
f. an NF1 loss of function mutation,
thereby treating the cancer.

2. The method according to claim 1, wherein said cancer is characterized by cells having
a. an NRAS mutation; and/or
b. a BRAF mutation; and/or
c. a cKIT mutation.

3. The method according to claim 1, wherein the NRAS mutation is selected from the group of an NRAS Q61 mutation, an NRAS G12 mutation and an NRAS G13 mutation, particularly wherein the NRAS mutation is selected from the group of NRAS Q61R, NRAS Q61K, NRAS Q61L, and NRAS Q61H, NRAS G12D, NRAS G12S, NRAS G12D, NRAS G12C, NRAS G12V, NRAS G12A, NRAS G13D, NRAS G13R, NRAS G13V, NRAS G13C, more particularly wherein the NRAS mutation is selected from the group of NRAS Q61K, NRAS Q61L, NRAS Q61R, NRAS Q61H, and NRAS G12A.

4. The method according to claim 1, wherein the KRAS mutation is selected from the group of a KRAS Q61 mutation, a KRAS G12 mutation, and a KRAS G13 mutation, particularly wherein the KRAS mutation is selected from the group of KRAS Q61R, KRAS Q61K, KRAS Q61L, KRAS Q61H, KRAS G13D, KRAS G13R, KRAS G13V, KRAS G13C, KRAS G12D, KRAS G12S, KRAS G12D, KRAS G12C, KRAS G12V, and KRAS G12A.

5. The method according to claim 1, wherein the HRAS mutation is selected from the group of an HRAS Q61 mutation, an HRAS G12 mutation, and an HRAS G13 mutation, particularly wherein the HRAS mutation is selected from the group of HRAS Q61R, HRAS Q61K, HRAS Q61L, HRAS Q61H, HRAS G12D, HRAS G12S, HRAS G12D, HRAS G12C, HRAS G12V, HRAS G12A, HRAS G13D, HRAS G13R, HRAS G13V, and HRAS G13C.

6. The method according to claim 1, wherein the BRAF mutation is selected from the group of BRAF V600E, BRAF V600K, BRAF V600D and BRAF V600R.

7. The method according to claim 1, wherein the cKIT mutation is selected from the group of cKIT K642E, cKIT L576P, cKIT V559A and cKIT W557R.

8. The method according to claim 1, wherein said cancer is characterized by cells that are resistant or refractory to treatment with a MEK inhibitor.

9. The method according to claim 1, wherein said cancer is characterized by cells that are resistant to treatment with a BRAF inhibitor.

10. The method according to claim 1, wherein said cancer is characterized by cells that are resistant to treatment with a cKJT inhibitor.

11. The method according to claim 1, wherein said cancer is characterized by cells that are resistant to treatment with panRAF inhibitor.

12. The method according to claim 1, wherein said cancer is characterized by cells that are resistant to treatment with a dual RAF/MEK inhibitor.

13. A method for treatment of a cancer comprising,
administering to a patient in need thereof a therapeutically effective amount of a combination medicament comprising or essentially consisting of
a. neocuproine; and
b. a second compound, wherein the second compound is a MAPK-pathway inhibitor;
thereby treating the cancer.

14. The method according to claim 13, wherein the second compound is selected from the group of a BRAF inhibitor, a panRAF inhibitor, a MEK inhibitor, and a dual RAF/MEK inhibitor.

15. The method according to claim 13, wherein the second compound is selected from the group of dabrafenib, encorafenib, vemurafenib, belvarafenib, selumetinib, binimetinib, cobimetinib, mirdametinib, pimasertib, selumetinib, trametinib, and avutometinib, particularly wherein the second compound is selected from the group of vemurafenib, encorafenib, dabrafenib, trametinib, binimetinib, combimetinib, mirdametinib, selumetinib.

16. A method for treatment of a cancer comprising,
administering to a patient in need thereof a therapeutically effective amount of a combination medicament comprising or essentially consisting of
a. neocuproine; and
b. a second compound, wherein the second compound is a tyrosine kinase inhibitor;
thereby treating the cancer.

17. The method according to claim 16, wherein the second compound is a cKJT inhibitor.

18. The method according to claim 16, wherein the second compound is selected from the group of imatinib, sunitinib, desatinib and nilotinib.

19. The method according to claim 1, wherein said cancer is selected from the group of melanoma, brain cancer, breast cancer, pancreatic cancer, lung cancer, and gastrointestinal cancer.

20. The method according to claim 1, wherein said cancer is selected from the group of melanoma, medulloblastoma, breast cancer, pancreatic cancer, glioblastoma, non-small cell lung cancer, and colon cancer, particularly wherein said cancer is melanoma.
